(19) United States
(12) Patent Application Publication
(10) Pub. No.: US 2025/0171829 A1
(43) Pub. Date: May 29, 2025

(54) MULTIPLEX FLUORESCENT CELLULAR AND TISSUE IMAGING WITH DNA ENCODED THERMAL CHANNELS AND USES THEREOF

(71) Applicant: PRESIDENT AND FELLOWS OF HARVARD COLLEGE, Cambridge, MA (US)

(72) Inventors: Fan HONG, Cambridge, MA (US); Jocelyn KISHI, Cambridge, MA (US); Sinem SAKA, Cambridge, MA (US); Jiyoun JEONG, Cambridge, MA (US); Peng YIN, Cambridge, MA (US)

(73) Assignee: PRESIDENT AND FELLOWS OF HARVARD COLLEGE, Cambridge, MA (US)

(21) Appl. No.: 18/844,272

(22) PCT Filed: Mar. 10, 2023
(86) PCT No.: PCT/US2023/014971

§ 371 Date: Sep. 5, 2024

Related U.S. Application Data
(60) Provisional application No. 63/319,021, filed on Mar. 11, 2022.

(51) Int. Cl. C12Q 1/6818; G01N 21/64

(52) U.S. Cl. CPC C12Q 1/6818; G01N 21/6458; G01N 2021/6432

(57) ABSTRACT

Provided herein are probes, methods and kits for detecting target molecule, e.g., in a multiplex manner at a single-cell or tissue level.

### CROSS-REFERENCE TO RELATED APPLICATIONS

This application is a 35 U.S.C. § 371 National Phase Entry Application of International Application No. PCT/US2023/014971 filed Mar. 10, 2023, which claims benefit under § 119(e) of U.S. Provisional Application No. 63/319,021 filed Mar. 11, 2022, the contents of both of which are incorporated herein by reference in their entireties.

### GOVERNMENT SUPPORT

This invention was made with government support under GM133052 and GM124401 awarded by the National Institutes of Health, and under N00014-18-1-2549 awarded by the U.S. Office of Naval Research. The government has certain rights in the invention.

### FIELD OF THE INVENTION

The field of the invention relates to the detection of target molecules, e.g., in a multiplex format.

### BACKGROUND

Spatial biology allows researchers to map the spatial architecture within a single cell and determine how it interacts with its soundings in a tissue environment. The spatial and quantitative information revealed at a single cell level dramatically expands the understanding of cell types in the human body and heterogeneity in a disease lesion micro-environment. Sequencing and imaging are the two major platforms for in situ spatial biology studies and can be performed without dissecting the cells. Compared with sequencing, imaging-based methods can give much higher spatial resolution in intact biological samples but typically suffer from low multiplexity of the biological targets analyzed. The ability to visualize many distinct species of biological molecules in single cells and tissue has become increasingly important tools to help understand complex biological systems, such as signal regulation pathways and cell heterogeneity in cancer tumor environments (Lewis, S. M. et al. Spatial omics and multiplexed imaging to explore cancer biology. Nature methods, 1-16 (2021))

However, the multiplexity of fluorescence microscopy is typically limited by the color pallet (generally 3˜4 colors) due to spectrum overlapping. The iterative binding of imagers and geometrical encoding of fluorophores on a larger entity has been developed to overcome this bottleneck. Such iterative methods require many rounds of washing to remove the previous round of signal and accumulate the current round of signal, which is achieved by the automated microfluidic system. The experiment setup is usually complicated, and the washing process is time-consuming. Geometrical encoding is achieved on a giant entity that is generally too bulky to diffuse to subcellular biological targets in situ to reveal high-resolution spatial information in a single cell. On top of those, combinational encoding can be applied to iterative binding or spectrum encoding to exponentially improve multiplexity (Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015) and Eng, C. L. et al. Transcriptome-scale super-resolved imaging in tissues by RNA seqFISH. Nature 568, 235-239 (2019)).

Thus, there remains a need in the art for reagents and methods for improved multiplex detection of target molecules (e.g., proteins and RNAs). The present disclosure addresses some of these needs.

### SUMMARY

The method, compositions and kits described herein are based, in part, on the generation of labelled nucleic acid probes with regions having different melting point/denaturation temperatures such that altering the temperature to a given temperature channel can release the inhibition of a reporter molecule, thereby permitting the visualization of a given nucleic acid in a cell or tissue.

In one aspect, provided herein is a method for detecting a target molecule in a sample. The method comprises providing a detection probe set. The detection probe set comprises at least three nucleic acid strands-a target binding strand, a reporter strand and a quencher strand. The first strand, also referred to as a target binding strand, comprises a target binding agent, for binding with the target, linked to a hybridization domain for hybridizing with the reporter strand. The second strand, also referred to as a reporter strand herein, comprises a first hybridization domain, for hybridizing with the hybridization domain of the target binding strand, linked to a second hybridization domain for binding with the quencher strand. The reporter strand further comprises a reporter molecule capable of producing a detectable signal. The first hybridization domain of the reporter strand comprises a nucleotide sequence that is substantially complementary to a nucleotide sequence of the hybridization domain of the target binding strand. The third strand, also referred to as a quencher strand, comprises a hybridization domain, for hybridizing with the second hybridization domain of the reporter strand. The quencher strand comprises a quencher molecule. The hybridization domain of the quencher strand comprises a nucleotide sequence that is substantially complementary to a nucleotide sequence of the second hybridization domain of the reporter strand. The reporter molecule in the reporter strand and the quencher molecule in the quencher strand are arranged such that the quencher molecule quenches the detectable signal from the reporter molecule when the reporter strand and the quencher strand are hybridized to each other, e.g., by the hybridization domains. When the quencher strand and the reporter strand are not hybridized to each other, the detectable signal from the reporter molecule is not quenched.

The target binding agent can be selected from the group consisting of nucleic acids, proteins, peptides, peptidomimetics, amino acids, disaccharides, trisaccharides, oligosaccharides, polysaccharides, lipopolysaccharides, lectins, nucleosides, nucleotides, vitamins, steroids, hormones, cofactors, receptors and receptor ligands. In some embodiments, the target binding agent is a nucleic acid, antibody, antigen binding fragment of an antibody, antibody mimetic, receptor, or a ligand for a receptor. For example, the target binding molecule is a nucleic acid. In another non-limiting example, the target binding molecule is an antibody or antigen binding fragment of an antibody.

The target molecules can be nucleic acids, proteins, saccharides, lipids, small molecules, or antigens. In some embodiments, the target molecule is a nucleic acid. For example, the target molecule is RNA, DNA or a mixture or RNA and DNA. In some embodiments, the target molecule is a protein. In some embodiments, the target molecule is a primary or secondary metabolite.

Melting temperatures of the different hybridization domains are different to allow release of the most quencher strand from report strand without substantially releasing the reporter strand from the target binding strand and/or release of the target binding strand from the target molecule. Generally, a melting temperature (Tm) of the quencher strand hybridizing with the reporter strand is lower than a melting temperature of the reporter strand hybridizing with the target binding strand, and a melting temperature of the quencher strand hybridizing with the reporter strand is lower than a melting temperature of the target strand binding with the target molecule. Generally, a melting temperature of the reporter strand hybridizing with the target binding strand is lower than target binding agent binding with the target molecule.

The probe set is contacted with the target under conditions to allow binding of the probe-set to the target by the binding of the target binding agent with the target. The binding of the probe-set to the target is analyzed by assessing a detectable signal produced as a function of temperature. For example, the detectable signal is detected or measured at a temperature higher than the melting temperature of the quencher strand hybridizing with the reporter strand and lower than the melting temperature of the reporter strand hybridizing with the target binding strand. At a temperature higher than the melting temperature of the quencher strand hybridizing with the reporter strand but lower than the melting temperature of the target binding agent binding to the target, the quencher strand and the reporter strand are no longer hybridized to each other and the detectable signal from the reporter molecule is not quenched. In contrast, when the detectable signal is measured at a temperature lower than the melting temperature of the quencher strand hybridizing with the reporter strand, the quencher strand and the reporter strand remain hybridizing with each other and the detectable signal from the reporter molecule is quenched. Thus, the detectable signal at the two temperature is different.

In some embodiments of the various aspects described herein, the target binding strand further comprises a second hybridization domain. The second hybridization domain of the target binding strand can be used to hybridize with a reference strand. The reference strand comprises a hybridization domain comprising a nucleotide sequence substantially complementary to a nucleic acid strand of the second hybridization domain of the target binding strand. The reference strand also comprises a reporter molecule capable of producing a detectable signal. A melting temperature of the reference hybridizing with the target binding strand is higher than the melting temperature strand of the reporter strand hybridizing with the target binding strand.

In some embodiments of any one of the aspects described herein, the detection probe set comprises at least four nucleic acid strands-a target binding strand, a reporter strand, a quencher strand and a reference strand. When a detection probe set comprising the four strands (a target binding strand, a reporter strand, a quencher strand and a reference strand) is used for detection, the method comprises detecting or measuring the detectable signal from the reporter molecules at a first temperature and a second temperature. The first temperature is a temperature higher than the melting temperature of the quencher strand hybridizing with the reporter strand but lower than the melting temperature of the reporter strand hybridizing with the target binding strand. The second temperature is a temperature higher than the melting temperature of the reporter strand hybridizing with the target binding strand but lower than the melting temperature of the reference strand hybridizing with the target binding strand. Detection of the detectable signal from the reporter molecule attached to the reference strand can serve as a control or reference.

In some embodiments, the method further comprises detecting or measuring the detectable signal from the reporter molecules at a temperature that is lower than the melting temperature of the reporter strand hybridizing with the target binding strand.

Different reporter and quencher strands can be prepared so that they have different melting temperature. Since the different quencher and reporter strand pairs denature or melt at different temperature, the detectable signal from different reporter molecules would be unquenched at different temperatures. Thus, binding of different probe sets to different targets can be assessed simultaneously under different temperatures. Accordingly, in another aspect provided herein is a method for multiplex detection of target molecules. The method comprises providing a plurality of detection probe sets, where each detection probe set comprises three strands—a target binding strand, a reporter strand and a quencher strand as described herein.

In the plurality of the probe sets, the melting temperature of the quencher strand hybridizing with the reporter strand in one detection probe set is different from the melting temperature of the quencher strand hybridizing with the reporter strand in at least one other probe set. Further, a melting temperature of the second strand in the probe set having the third strand with the lower melting temperature is about same or lower than a melting temperature of the second strand in the probe set having the third strand with the higher melting temperature. It is noted that a quencher strand in one detection probe set may or may not be capable of hybridizing with a reporter strand in another detection probe set in the plurality of the detection probe sets.

The plurality of the detection probe sets is contact with the target molecules to allow binding of the detection probe-sets to their respective targets by the binding of the target binding agent with the target. The binding of the detection probe-sets to the targets is analyzed by assessing a detectable signal produced as a function of temperature. For example, a detectable signal from a first probe-set binding with its target is measured at a temperature higher than the melting temperature of the quencher strand hybridizing with the reporter strand and lower than the melting temperature of the reporter strand hybridizing with the target binding strand. The temperature is such that the quencher strand in a second detection probe set that is bound to a target remains hybridized with the reporter strand in that second detection probe set. A detectable signal from the second probe-set binding with its target is measured at a temperature higher than the melting temperature of quencher strand of the second probe set hybridizing with the reporter strand of the second probe set and lower than the melting temperature of reporter strand of the second probe set hybridizing with the target binding strand of the second probe set. As the detectable signals from different probe-sets are generated at different temperature, binding of different detection probe sets to their respective targets can be distinguished and targets detect in a multiplex manner. This allows for the detection of multiple target molecules in one reaction, i.e., multiplexed detection. In some embodiments, at least two target molecules are detected, e.g., at least 2, at least 3, at least 4, at least 5, at least 6, at least 7. at least 8, at least 9, at least 10 or more different target molecules are detected.

It is noted that the reporter molecules in the different members of the plurality of the detection probe sets can be same or different. Accordingly, in some embodiments of the various aspects described herein, the reporter molecule in one detection probe set in the plurality is different from the reporter molecule in at least one other detection probe set in the plurality. Without wishing to be bound by theory, use of different reporter molecule can provide additional multiplex detection capabilities. For example, probe sets where the melting temperature of the quencher strand hybridizing with the reporter strand are similar but the different probe sets have different reporter molecules can be used. The detectable signal from one reporter molecule is distinguishable from the detectable signal from at least one reporter molecule. The distinguishable detectable signals can be measured at one temperature to detect binding of the different probe sets to their respective targets, e.g., a temperature higher than the melting temperature of the quencher strand hybridizing with the report strand. This allows for the detection of multiple target molecules in one reaction, i.e., multiplexed detection. In some embodiments, at least two target molecules are detected, e.g, at least 2, at least 3, at least 4, at least 5, at least 6, at least 7, at least 8, at least 9, at least 10 or more different target molecules are detected. In some embodiments, each target molecule is detected with a detection probe set comprising a distinguishable reporter molecule. These can be used with other quencher and reporter strand pairs that denture or melt at a different temperature for further multiplexing.

In another aspect, provided herein is a detection probe set. The detection probe set comprises at least three nucleic acid strands-a target binding strand, a reporter strand and a quencher strand as described herein.

In yet another aspect, provided herein a kit. The kit comprises a detection probe set as described herein. In some embodiments, the kit comprises a plurality of detection probe sets as described herein.

It is noted that the detection of the detectable label can be performed with fluorescence microscopy and fluoropoher-labeled reporters. Or with colorimetric detection and e.g. nanoparticle-labeled reporters. Detection can also be done for multiplexed imaging of biological species in fixed cell, organoid, or tissue samples of prokaryotic, eukaryotic, other, or mixed origin. Readout can also be performed on a surface e.g. glass slide or other substrate, such as for sequencing of barcoded beads on a surface or for sequencing of immobilized oligos on a surface for nucleic acid data storage applications.

There are multiple ways samples can be brought to the correct temperature. One way is with a Peltier on-stage system such as VAHEAT. Another is to have a buffer that is continuously flowed through the chamber where an external heater controls the buffer temperature. Another way to control temperature is by using lasers or light coupled with metallic nanomaterials to convert light to thermal energy. See, for example, www.cell.com/trends/biochemical-sciences/fulltext/S0968-0004 (19) 30236-1?dgcid.

### BRIEF DESCRIPTION OF THE DRAWINGS

FIGS. 1A-1C. The scheme of thermal multiplexed imaging according to an exemplary embodiment. (FIG. 1A) The imaging concept and scheme of the DNA thermal-scope; (FIG. 1B) The scheme of thermal control platform with cells or tissues; (FIG. 1C) The thermal-spectrum of the DNA thermal-scope.

FIGS. 2A-2C. Programmable thermal spectrums of the DNA thermal scope. (FIG. 2A) The signal yield of thermal spectrum with different combination of quencher and binder's melting temperature. (FIG. 2B) The signal yield of thermal spectrum with different combination of quencher and binder's melting temperature. (FIG. 2C) The thermal spectrum of five optimal thermal channels with minimal crosstalk and high signal yield.

FIGS. 3A-3E. The robustness and fast channel switching speed of DNA thermal scope. (FIG. 3A) RNA imaging process with reference probes and thermal probes at different temperature channels. (FIG. 3B) The imaging of APC RNA transcript in Hela cells in situ. (FIG. 3C) The puncta analysis of the reference probe and thermal probe-based imaging. (FIG. 3D) The fluorescent signal of the puncta detected by DNA thermal scope at different heating time at three temperature channels. (FIG. 3E) The FISH images of APC gene after heating at different temperature channel for 5 s.

FIGS. 4A-4B. The design of five thermal channels with minimal signal crosstalk. (FIG. 4A) The FISH images for different designed DNA thermal probes at different temperature channels. (FIG. 4B) The average RNA number detected per cell with different DNA thermal probes at different temperature channels.

FIGS. 5A-5C. 15-plex RNA imaging with the combination of thermal channels and fluorophore channels in fixed Hela Cells. (FIG. 5A) The imaging process of DNA thermal scope at different temperature channels. (FIG. 5B) The individual RNA puncta at different combination of DNA thermal channels and fluorophore channels. (FIG. 5C) The overlapped imaging of 15 imaged RNA species in single cell.

FIGS. 6A-6B. 15-plex RNA imaging in retina tissue with thermal-plex. (FIG. 6A) The workflow of DNA thermal scope at different temperature channels. (FIG. 6B) The individual RNA puncta at different imaging of 15 imaged RNA species in a single cell.

FIGS. 7A-7B. Resolved 15-plex RNA information. (FIG. 7A) Reconstructed 15-plex RNA imaging in retina tissue. FIG. 7B) The comparison between smFISH and thermal-plex imaging for 15 RNA targets.

FIGS. 8A-8E are schematic of thermal-plex imaging. (FIG. 8A) The thermal-plex imaging concept is based on stepwise melting of DNA thermal probes from an in situ target. After labeling the target biomolecule (e.g, DNA, RNA or proteins) with a DNA barcode, the thermal probe set comprising a quencher strand and an imager strand is hybridized to the DNA barcode which is attached to the target. At temperatures lower than the melting temperature of the quencher, the fluorescent signal is quenched. When the temperature is heated to the signal temperature, the quencher is melted off and fluorescence signal is emitted and can be imaged, including after cooling the sample to room temperature. Signal is then removed by heating to a temperature substantially higher than the melting temperature of the barcode domain (to which the imager binds). (FIG. 8B) The thermal spectrum of an exemplary thermal-plex probe set. The melting profiles of quencher and imager pairs are used to determine the thermal profile of a DNA thermal probe set. Tₛ is the temperature that gives maximum fluorescent signal. T_{mq} is the melting temperature of the quencher. T_{mb} is the melting temperature of the barcode domain. The width is the distance between the half maximum of the signal. (FIG. 8C) The imaging setup for thermal-plex. Cells are seeded on a slide embedded within an on-scope temperature control device, which can rapidly change the slide temperature in seconds. The volumetric buffer allows effective DNA strand dissociation to activate or remove the fluorescence signal after heating spike. (FIG. 8D) The temperature profile of the slide during the heating and cooling process. It takes about 5 seconds to reach the desired signal temperature (57° C.) to melt off the DNA strands. After the temperature is cooled down to around 30° C., the sample is imaged. (FIG. 8E) Example process of multiplexed fluorescent imaging for three targets (Ta, Tb, and Tc) with thermal-plex. (1) Single-stranded DNA barcodes (a1, , b1, and c1) are attached to different target molecules. (2) Orthogonal DNA thermal probe sets (a, b, c) are hybridized to the DNA barcodes. (3) The target Ta is visualized at room temperature after the heating to signal temperature Tₛₐ to activate the fluorescence signal. Multiple other targets are visualized sequentially after heating to their assigned signal temperatures (T_{sb}, T_{sc}). (4) The images are computationally aligned and reconstructed to overlay all the targets.

FIG. 9 is a schematic showing multiplexed protein imaging in tissue samples with thermal-plex imaging according to an exemplary embodiment. Target proteins are firstly bind with its corresponding antibody conjugated with DNA barcodes. DNA thermal probes encode different thermal channels will be used bind to the DNA barcodes on the antibody. Multi-rounds of heating spike at different temperatures and imaging are applied to activate the signal of the DNA thermal probes.

FIG. 10 is a schematic showing thermal-plex for protein imaging according to an exemplary embodiment. DNA-barcode conjugated antibody is firstly applied to bind to the target proteins. DNA thermal probes are then bind to the DNA barcode. Heating and imaging is used to visualize the target proteins.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Provided herein are nucleic acid probe compositions that permit differential detection of targets in a single-cell or tissue using temperature-sensitive melting probes that are detectable only upon exposure to a particular threshold temperature or range of temperatures (e.g., a thermal channel).

Embodiments of the various aspects described herein include a detection probe set. The detection probe set comprises at least three nucleic acid strands-a target binding strand, a reporter strand and a quencher strand. As described herein, the quencher strand and reporter strands are capable of hybridizing with each other and the reporter strand and the target binding strand are capable of hybridizing with each other. Thus, in some embodiments of any one of the aspects described herein, the quencher strand and reporter strands are hybridized with each other and/or the reporter strand and the target binding strand are hybridized with each other.

As used herein, the term “hybridize” refers to the interaction or annealing of two single-stranded nucleic acids into a double-stranded nucleic acid (e.g., duplex) under specific hybridization conditions. Typically, two single-stranded nucleic acids form a duplex when they have sufficient complementarity to each other under a give set of conditions Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

### Target Binding Strand

Embodiments of the various aspects described herein include a target binding strand. Generally, the target binding strand comprises a target binding agent linked to a first hybridization domain.

The length of the first hybridization domain of the target binding strand can be in the range of, for example, about 10 to about 100 nucleotides, or about 15 to about 90 nucleotides, about 20 to about 80 nucleotides or about 25 to about 75 nucleotides or about 30 to about 50 nucleotides in length. Generally, the length of the first hybridization domain is chosen such that a melting temperature of the first hybridization domain hybridizing with a complementary strand is lower than the temperature at which the target binding agent dissociates from its target. For example, the melting temperature of the first hybridization domain hybridizing with a complementary strand, e.g., a reporter strand is at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. lower compared to the temperature at which the target binding agent dissociates from its target.

In some embodiments, the melting temperature of the first hybridization domain hybridizing with a complementary strand, e.g., a reporter strand is at least 5° C. lower compared to the temperature at which the target binding agent dissociates from its target

The target binding strand also comprises a target binding agent, also referred to as a target binding domain herein. It is noted that the target binding agent and first hybridization domain can be arranged in any desired orientation in the target binding strand. For example, the target binding agent can be at the 5′-end of the first hybridization domain or at the 3′-end of the first hybridization domain. In some embodiments, the target binding agent is at the 5′-end of first the hybridization domain. In some other embodiments, the target binding agent is at the 3′-end of the first hybridization domain. In some embodiments, the target binding agent is a nucleic acid. For example, the target binding agent is a single-stranded nucleic acid comprising a nucleotide sequence that is substantially complementary to at least a portion of a target nucleic acid. When the target binding agent is a nucleic acid, the length of the target agent nucleic acid strand can be in the range of, for example, about 25 to about 200 nucleotides, or about 30 to about 150 nucleotides, about 35 to about 125 nucleotides or about 40 to about 100 nucleotides or about 50 to about 75 nucleotides in length.

In some embodiments, the target binding agent is an antibody or an antigen binding portion of an antibody. Generally, the target binding agent is chosen such that a melting temperature of the target binding agent binding or hybridizing with the target is higher than the melting of the hybridizing domain hybridizing with the reporter strand. For example, the melting temperature of the target binding agent binding or hybridizing with the target is at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. higher compared to the melting temperature of the hybridization domain hybridizing with the reporter strand.

In some embodiments of any one of the aspects described herein, the melting temperature of the target binding agent binding or hybridizing with the target is at least 5° C. higher compared to the melting temperature of the hybridization domain hybridizing with the reporter strand.

In some embodiments of any one of the aspects described herein, when the target binding agent is a nucleic acid, the length of the target binding nucleic acid is longer than the length of the hybridization domain of the target binding strand.

In some embodiments, the target binding strand further comprises a second hybridization domain, e.g., a hybridization domain for hybridizing with a reference nucleic acid strand. This domain is also referred to as a reference binding domain herein. The length of the reference binding domain can be in the range of, for example, about 25 to about 200 nucleotides, or about 30 to about 150 nucleotides, about 35 to about 125 nucleotides or about 40 to about 100 nucleotides or about 50 to about 75 nucleotides in length. Generally, the length of the reference binding domain is chosen such that a melting temperature of the reference binding domain hybridizing with a complementary reference strand is higher than the melting of the hybridizing domain hybridizing of the target binding strand with the reporter strand. For example, the melting temperature of the reference binding domain hybridizing with a complementary reference strand is at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. higher compared to the melting temperature of the hybridization domain of the target binding strand hybridizing with the reporter strand.

In some embodiments of any one of the aspects described herein, the melting temperature of the reference binding domain hybridizing with a complementary reference strand is at least 5° C. higher compared to the melting temperature of the hybridization domain of the target binding strand hybridizing with the reporter strand.

The reference binding domain can be located anywhere in the target binding strand. In some embodiments, the reference binding domain is at the 5′-end of the target binding strand. In some other embodiments, the reference binding domain is at the 3′-end of the target binding strand. In some embodiments, the target binding strand comprises in a 5′->3′ direction, the reference binding domain linked to the target binding agent that is linked to the first hybridization domain. In some other embodiments, the target binding strand comprises in a 5′->3′ direction, the first hybridization domain linked to the target binding agent that is linked to the reference binding domain.

### Reporter Strand

Embodiments of the various aspects described herein include a reporter nucleic acid strand. Generally, the reporter nucleic acid strand comprises a first hybridization domain linked to a second hybridization domain, and a reporter molecule is linked to the reporter strand. The first hybridization domain comprises a nucleotide sequence substantially complementary to a nucleotide sequence of the first hybridization domain of the target binding strand.

The length of the first hybridization domain of the reporter strand can be same or different than the length of the first hybridization domain of the target binding strand. For example, the length of the first hybridization domain of the reporter strand can be in the range of, for example, about 10 to about 100 nucleotides, or about 15 to about 90 nucleotides, about 20 to about 80 nucleotides or about 25 to about 75 nucleotides or about 30 to about 50 nucleotides in length. Generally, the length of the first hybridization domain of the report strand is chosen such that a melting temperature of the reporter stand hybridizing with the target binding strand is lower than the temperature at which the target binding agent dissociates from its target. For example, the melting temperature of the first hybridization domain of the reporter stand hybridizing with the first hybridization domain of the target binding strand is at least 2° C., at least 3° C., at least 4° C., at least 5° C., at least 6° C., at least 7° C., 8° C., at least 9° C., at least 10° C., at least 11° C., at least 12° C., at least 13° C., at least 14° C., or at least 15° C. lower compared to the temperature at which the target binding agent dissociates from its target.

In some embodiments of any one of the aspects described herein, the melting temperature of the first hybridization domain of the reporter stand hybridizing with the first hybridization domain of the target binding strand is at least 5° C. lower compared to the temperature at which the target binding agent dissociates from its target.

The length of the second hybridization domain of the reporter strand can be same or different than the length of the first hybridization domain of the reporter strand. For example, the length of the second hybridization domain of the reporter strand can be in the range of, for example, about 10 to about 100 nucleotides, or about 15 to about 90 nucleotides, about 20 to about 80 nucleotides or about 25 to about 75 nucleotides or about 30 to about 50 nucleotides in length. Generally, the length of the second hybridization domain of the report strand is chosen such that a melting temperature of the reporter stand hybridizing with the quencher strand is lower than the melting temperature of the reporter stand hybridizing with the target binding strand. For example, the melting temperature of the second hybridization domain of the reporter stand hybridizing with a complementary strand, e.g., a quencher strand is at least 2° C., at least 3° C., at least 4° C., at least 5° C., at least 6° C., at least 7° C., 8° C., at least 9° C., at least 10° C., at least 11° C., at least 12° C., at least 13° C., at least 14° C., or at least 15° C. lower compared to the melting temperature of the reporter stand hybridizing with the target binding strand.

In some embodiments of any one of the aspects described herein, the melting temperature of the second hybridization domain of the reporter stand hybridizing with a complementary strand, e.g., a quencher strand is at least 5° C. lower compared to the melting temperature of the reporter stand hybridizing with the target binding strand.

It is noted that the first and second hybridization domains of the reporter strand can be arranged in any desired orientation. For example, the first hybridization domain of the reporter strand can be at the 5′-end or 3′-end of the second hybridization domain of the reporter strand. Accordingly, in some embodiments, the first hybridization domain of the reporter strand is at the 5′-end of the second hybridization domain of the reporter strand. In some other embodiments, the first hybridization domain of the reporter strand is at the 3′-end of the second hybridization domain of the reporter strand.

The reporter strand also comprises a reporter molecule that is capable of generating a detectable signal. Generally, the reporter molecule is located near or in the second hybridization domain of the reporter strand such that hybridizing the reporter strand with the quencher strand quenches the detectable signal.

### Quencher Strand

Embodiments of the various aspects described herein include a quencher nucleic acid strand. Generally, the quencher nucleic acid strand comprises a first hybridization domain and a quencher molecule. The first hybridization domain comprises a nucleotide sequence substantially complementary to a nucleotide sequence of the second hybridization domain of the reporter strand.

The length of the first hybridization domain of the quencher strand can be same or different than the length of the second hybridization domain of the reporter strand. For example, the length of the first hybridization domain of the quencher strand can be in the range of, for example, about 10 to about 100 nucleotides, or about 15 to about 90 nucleotides, about 20 to about 80 nucleotides or about 25 to about 75 nucleotides or about 30 to about 50 nucleotides in length. Generally, the length of the first hybridization domain of the quencher strand is chosen such that a melting temperature of the quencher stand hybridizing with the reporter strand is lower than the melting temperature of the reporter strand hybridizing with the target binding strand. For example, the melting temperature of the first hybridization domain of the quencher stand hybridizing with the second hybridization domain of the reporter strand is at least 2° C., at least 3° C., at least 4° C., at least 5° C., at least 6° C., at least 7° C., 8° C., at least 9° C., at least 10° C., at least 11° C., at least 12° C., at least 13° C., at least 14° C., or at least 15° C. lower compared to the melting temperature of the reporter strand hybridizing with the target binding strand.

In some embodiments of any one of the aspects described herein, the melting temperature of the first hybridization domain of the quencher stand hybridizing with the second hybridization domain of the reporter strand is at least 5° C. lower compared to the melting temperature of the reporter strand hybridizing with the target binding strand.

The quencher strand also comprises a quencher molecule that is capable of quenching a detectable signal from the reporter molecule. Generally, the quencher molecule in the quencher strand is located such that the detectable signal from the reporter molecule is quenched when the quencher strand and the reporter strand are hybridized to each other. When the quencher strand and the reporter strand are not hybridized to each other, the detectable signal is not quenched. In some embodiments, the quencher molecule is one member of a FRET pair. For example, the quencher can be a FRET accepter.

### Reference Strand

Embodiments of the various aspects described herein include a reference nucleic acid strand. Generally, the reference nucleic acid strand comprises a first hybridization domain and a reporter molecule capable of generating a detectable signal. The first hybridization domain comprises a nucleotide sequence substantially complementary to a nucleotide sequence of the second hybridization domain of the target binding strand.

The length of the first hybridization domain of the reference strand can be same or different than the length of the second hybridization domain of the target binding strand. The length of the first hybridization domain of the reference strand can be in the range of, for example, about 25 to about 200 nucleotides, or about 30 to about 150 nucleotides, about 35 to about 125 nucleotides or about 40 to about 100 nucleotides or about 50 to about 75 nucleotides in length. Generally, the length of the first hybridization domain of the reference strand is chosen such that a melting temperature of the reference strand hybridizing with the target binding strand is higher than the melting temperature of the target binding strand hybridizing with the reporter strand. For example, the first hybridization domain of the reference strand hybridizing with the second hybridization domain of the target binding strand is at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. higher compared to the melting temperature of the target binding strand hybridizing with the reporter strand.

In some embodiments of any one of the aspects described herein, the first hybridization domain of the reference strand hybridizing with the second hybridization domain of the target binding strand is at least 5° C. higher compared to the melting temperature of the target binding strand hybridizing with the reporter strand.

As described herein, melting temperatures can be different between the various hybridization domains for hybridizing their complementary strands. Generally, the melting temperature of the quencher strand hybridizing with the reporter strand is lower than the melting temperature of the reporter strand hybridizing with the target binding strand, and the the melting temperature of the reported strand hybridizing with the target binding strand is lower than the temperature at which the target binding agent dissociates from the target, i.e., lower than the melting temperature of target strand hybridizing with the target molecule.

### Reporter Molecule

Embodiments of the various aspects described herein rely on a reporter molecule capable of producing a detectable signal. For example, the reporter strand comprises a reporter molecule. As used herein, the terms “reporter molecule” and “detectable label” are used interchangeably and can refer to any chemical moiety attached to a nucleotide, nucleotide polymer, or nucleic acid binding factor, wherein the attachment can be covalent or non-covalent and permits the detection of a signal in a single cell or tissue. Reporter molecules can include luminescent molecules, chemiluminescent molecules, fluorochromes, fluorescent quenching agents, colored molecules, radioisotopes or scintillants Reporter molecules can also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. The skilled artisan would readily recognize useful reporter molecules that are not mentioned above, which may be employed in the methods and compositions described herein. In one embodiment, the reporter molecule comprises a fluorophore portion of a fluorescence resonance energy transfer (FRET) pair.

In some embodiments of any of the aspects, the reporter molecule provided herein is selected from the group consisting of: fluorescent molecules/fluorophores, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

In some embodiments of any one of the aspects described herein, the reporter molecule is a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore A wide variety of fluorescent reporter dyes are known in the art Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS: 1,8-ANS; 4-Methylumbelliferone; S-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (S-Carboxytetramethylrhodamine), 6-Carboxyrhodamine 6G; 6-CR. 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow, Acriflavin; Acriflavin Feulgen SITSA: Aequorin (Photoprotein); Alexa Fluor 350™; Alexa Fluor 430™; Alexa Fluor 488™; Alexa Fluor 532™; Alexa Fluor 546™, Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X: Aminoactinomycin D; Aminocoumarin; Anilin Blue; Anthrocyl stearate, APC-Cy7, APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1, BOBO™-3; Bodipy 492/515, Bodipy 493/503; Bodipy 500/510; Bodipy 505/515; Bodipy 530/550; Bodipy 542/563; Bodipy 558/568; Bodipy 564/570, Bodipy 576/589; Bodipy 581/591; Bodipy 630/650-X, Bodipy 650/665-X; Bodipy 665/676; Bodipy Fl; Bodipy FL ATP; Bodipy FI-Ceramide; Bodipy R6G SE; Bodipy TMR: Bodipy TMR-X conjugate; Bodipy TMR-X, SE; Bodipy TR, Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A, Chromomycin A; CMFDA; Coelenterazine; Coelenterazine cp: Coelenterazine f; Coelenterazine fop; Coelenterazine h; Coelenterazine bcp; Coclenterazine ip, Coelenterazine O, Coumarin Phalloidin, CPM Methylcoumarin; CTC; Cy2™; Cy3.1 8; Cy3.5™; Cy3™; Cy5.1 8; Cy5.5™; Cy5™; Cy 7™; Cyan GFP, cyclic AMP Fluorosensor (FiCRhR); d2; Dabcyl, Dansyl; Dansyl Amine; Dansyl Cadaverine, Dansyl Chloride; Dansyl DHPE, Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DIO (DiOC18 (3)); DIR; DIR (DiIC18 (7)); Dopamine; DsRed; DTAF; DY-630-NHS, DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP, Fast Blue, FDA; Feulgen (Pararosaniline); FITC, FL-645; Flazo Orange; Fluo-3; Fluo-4; Fluorescein Diacetate; Fluoro-Emerald: Fluoro-Gold (Hydroxystilbamidine); Fluor-Ruby; FluorX; FM 1-43™; FM 4-46; Fura Red™M (high pH); Fura-2, high calcium; Fura-2, low calcium, Genacryl Brilliant Red B; Genacryl Brilliant Yellow 10GF; Genacryl Pink 3G; Genacryl Yellow SGF; GFP (S65T); GFP red shifted (rsGFP), GFP wild type, non-UV excitation (wtGFP), GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DID): Indotricarbocyanine (DiR); Intrawhite Cf; JC-1; JO-JO-1; JO-PRO-1, LaserPro; Laurodan; LDS 751; Leucophor PAF: Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue, Maxilon Brilliant Flavin 10 GFF; Maxilon Brilliant Flavin 8 GFF; Merocyanin; Methoxycoumarin; Mitotracker Green FM; Mitotracker Orange; Mitotracker Red, Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole, Noradrenaline, Nuclear Fast Red; Nuclear Yellow; Nylosan Brilliant lavin ESG; Oregon Green™; Oregon Green 488-X; Oregon Green™ 488; Oregon Green™ 500, Oregon Green™ 514; Pacific Blue; Pararosaniline (Feulgen), PE-CyS; PE-Cy7; PerCP: PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]: Phycoerythrin R [PE]; PKH26; PKH67, PMIA, Pontochrome Blue Black; POPO-1; POPO-3; PO-PRO-1; PO-PRO-3; Primuline; Procion Yellow; Propidium Iodid (PI), PyMPO; Pyrene, Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD, Rhodamine 6G; Rhodamine B 540; Rhodamine B 200; Rhodamine B extra; Rhodamine BB; Rhodamine BG: Rhodamine Green; Rhodamine Phallicidine; Rhodamine Phalloidine; Rhodamine Red; Rhodamine WT; Rose Bengal; R-phycoerythrin (PE), red shifted GFP (rsGFP, S65T): S6SA; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine: Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange: Tinopol CBS (Calcofluor White); TMR; TO-PRO-1; TO-PRO-3; TO-PRO-5; TOTO-1; TOTO-3; TriColor (PE-CyS); TRITC (TetramethylRodaminelsoThioCyanate); True Blue; TruRed, Ultralite; Uranine B; Uvitex SFC; wt GFP; WW 781; XL665; X-Rhodamine; XRITC; Xylene Orange; Y66F; Y66H; Y66W; Yellow GFP, YFP; YO-PRO-1; YO-PRO-3, YOYO-1; and YOYO-3 Many suitable forms of these fluorescent compounds are available and can be used.

Many suitable forms of these fluorescent compounds are available and can be used. Additional fluorophore examples include, but are not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-CyS™, green fluorescent protein, rhodamine, fluorescein isothiocyanate (FITC) and Oregon Green™, rhodamine and derivatives (e.g., Texas red and tetramethylrhodamine isothiocyanate (TRITC)), biotin, phycoerythrin, AMCA, CyDyes™, 6-carboxyfluorescein (commonly known by the abbreviations FAM and F), 6-carboxy-2′, 4′,7′,4,7-hexachlorofluorescein (HEX), 6-carboxy-4′,5′-dichloro-2′, 7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110, cyanine dyes, e.g Cy3, Cy5 and Cy7 dyes; coumarins, e.g., umbelliferone: benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g, cyanine dyes such as Cy3, Cy5, etc.; BODIPY dyes and quinoline dyes.

Other exemplary detectable labels include luminescent and bioluminescent markers (e.g., biotin, luciferase (e.g., bacterial, firefly, click beetle and the like), luciferin, and aequorin), radiolabels (e.g, 3H, 125I, 35S, 14C, or 32P), enzymes (e.g, galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference

In some embodiments of any of the aspects, a detectable label can be a radiolabel including, but not limited to 3H, 1251, 35S, 14C, 32P, and 33P Suitable non-metallic isotopes include, but are not limited to, IIC, 14C, 13N, 18F. 1231. 1241. and 1251. Suitable radioisotopes include, but are not limited to, 99mTc, 95Tc, 111 In, 62Cu, 64Cu, Ga, 68Ga, and 153Gd. Suitable paramagnetic metal ions include, but are not limited to, Gd(III), Dy(III), Fe (III), and Mn (II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the detectable label is a fluorophore or a quantum dot. Without wishing to be bound by a theory, using a fluorescent reagent can reduce signal-to-noise in the imaging/readout, thus maintaining sensitivity.

### Quencher molecule

The advantage of the probes described herein is that inhibition of a reporter molecule is released only within a certain thermal channel (e.g., range of temperatures) due to a denaturation-mediated removal of a quencher molecule. Multiple reporter molecules and their corresponding quencher pair can be designed to denature and induce fluorescence at multiple different thermal channels.

Accordingly, embodiments of the various aspects described herein include a quencher molecule. For example, the quencher strand comprises a quencher molecule. The quencher can act to decrease a detectable property, e.g., the intensity, color, etc. of the detectable signal from a reporter molecule provided herein. In some embodiments, the quencher molecule is at the 5′ end of the quencher strand. In some embodiments, the quencher molecule is at the 3′ end of the quencher strand. In some embodiments, the quencher molecule is at an internal position of the quencher strand. Generally, the quencher molecule is positioned in the quencher strand such that when the quencher strand and the reporter strand hybridize with each other a detectable property, e.g., the intensity, color, etc. of the detectable signal from a reporter molecule is decreased or quenched. And, when the quencher strand and the reporter strand are not hybridized with each other the detectable property, e.g., the intensity, color, etc. of the detectable signal from a reporter molecule is not quenched or decreased. Multiple quencher molecules can be used, for example, in order to quench multiple different reporter molecules. In some embodiments, the nucleic acid probe comprises 2, 3, 4, 5 or more quencher molecules, which can be the same or different from each other.

In some embodiments of any of the aspects, the quenching is partial quenching or complete quenching. As used herein the term “completely quenched” refers to the inability to detect any signal from the reporter molecule, i.e., 100% quenched or 0% detectable signal (e.g., fluorescence). As used herein the term “partially quenched” refers to a detectable signal from the reporter molecule that is reduced compared to the full detectable signal from the reporter molecule. In some embodiments of any of the aspects, “partially quenched” refers to a signal from the reporter molecule that is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.5%, at least 99.9% or more.

In some embodiments of any of the aspects, the at least one quencher molecule quenches the specific wavelength of the fluorescence emitted by the reporter molecule in the reporter probe. As a non-limiting example, some fluorophores, such as TET, HEX, and FAM, with an emission range between 500 nm to 550 nm are quenched by quenchers, such as Black hole quencher 1 (BHQ1) and Dabcyl, with an absorption range of 450 nm to 550 nm. Similarly, TMR, Texas red, ROX, Cy3, and Cy5 are quenched by BHQ2. See e.g., Marras, Selection of fluorophore and quencher pairs for fluorescent nucleic acid hybridization probes, Methods Mol Biol. 2006;335:3-16; the content of which is incorporated herein by reference in its entirety.

In some embodiments of any of the aspects, the quencher molecule is a dark quencher. A dark quencher (also known as a dark sucker) is a substance that absorbs excitation energy from a reporter molecule, e.g., a fluorophore, and dissipates the energy as heat; while a typical (fluorescent) quencher re-emits much of this energy as light. Non-limiting examples of quencher molecules (e.g., non-fluorescent or dark quenchers that dissipate energy absorbed from a fluorescent dye) include the Black Hole Quenchers™ (Biosearch Technologies™); Iowa Black quenchers (e.g., Iowa Black FQ™ (“3IABKFQ”) and Iowa Black RQ™ (e.g., “3IAbRQSp”)); Eclipse® Dark Quenchers (Epoch Biosciences™), Zen™ quenchers (Integrated DNA Technologies™; “e.g., “ZEN”); TAO™ quenchers (Integrated DNA Technologies™; “e.g., “TAO”); Dabcyl (4-(4′-dimethylaminophenylazo) benzoic acid); Qxl™ quenchers; QSY® quenchers; and IRDye® QC-1. Additional non-limiting examples of quenchers are also provided in U.S. Pat. No. 6,465,175, 7,439,341, 12/252,721, 7,803,536, 12/853,755, 7,476,735, 7,605,243, 7,645,872, 8,030,460, 13/224,571, 8,916,345, the contents of each of which are incorporated herein by reference in their entireties.

In some embodiments of any of the aspects, the quencher molecule is an Iowa Black® quencher. In some embodiments of any of the aspects, the Iowa Black® quencher is preferably at the 5′ or 3′ position of the nucleic acid probe. In some embodiments of any of the aspects, the quencher molecule is Iowa Black® FQ, which has a broad absorbance spectra ranging from 420 to 620 nm with peak absorbance at 531 nm (i.e., the green-yellow region of the visible light spectrum). In some embodiments, Iowa Black® FQ (e.g., “3IABKFQ”) is used to quench fluorescein or other fluorescent dyes that emit in the green to pink spectral range. In some embodiments of any of the aspects, the quencher molecule is Iowa Black® RQ, which has a broad absorbance spectra ranging from 500 to 700 nm with peak absorbance at 656 nm (i.e., the orange-red region of the visible light spectrum). In some embodiments, Iowa Black® RQ (e.g., “3IAbRQSp”) is used to quench Texas Red®, Cy5, or other fluorescent dyes that emit in the red spectral range.

In some embodiments of any of the aspects, the quencher molecule is a ZEN quencher. In some embodiments of any of the aspects, the ZEN quencher is preferably at an internal position of the nucleic acid probe. See e.g., Lennox et al., Mol Ther Nucleic Acids. 2013 August; 2 (8): e117; U.S. Pat. Nos. 8,916,345, 9506059; the contents of each of which are incorporated herein by reference in their entireties. ZEN can quench a similar range of fluorophores as Iowa Black® FQ, e.g., FAM, SUN, JOE, HEX, or MAX. In some embodiments, the nucleic acid probe comprises ZEN, Iowa Black® FQ, and a reporter molecule such as FAM.

In some embodiments of any of the aspects, the quencher molecule is a TAO quencher. In some embodiments of any of the aspects, the TAO quencher is preferably at an internal position of the nucleic acid probe. TAO can quench a similar range of fluorophores as Iowa Black® RQ, e.g., Cy3, ATTO550, ROX, Texas red, ATTO647N, or Cy5. In some embodiments, the nucleic acid probe comprises TAO, Iowa Black® RQ, and a reporter molecule, such as Cy5.

In some embodiments of any of the aspects, the quencher molecule is a black hole quencher. The Black Hole Quenchers™ are structures comprising at least three radicals selected from substituted or unsubstituted aryl or heteroaryl compounds, or combinations thereof, wherein at least two of the residues are linked via an exocyclic diazo bond (see, e.g., International Publication No. WO2001086001). Black Hole Quenchers (BHQ) are capable of quenching across the entire visible spectrum. Non-limiting examples of Black Hole Quenchers include BHQ-0 (430-520 nm); BHQ-1 (480-580 nm, 534 nm absorbance (abs) max); BHQ-2 (520-650 nm, 544 nm abs max); BHQ-3 (620-730 nm, 672 nm abs max); and BHQ-10 (480-550nm, 516 nm abs max; Water Soluble).

In some embodiments of any of the aspects, the quencher molecule is Dabcyl (4-(4′-dimethylaminophenylazo) benzoic acid) or a derivative thereof. Dabcyl absorbs in the green region of the visible light spectrum (e.g., 346-489 nm, with a peak absorbance at 474 nm) and can be used with fluorescein or other fluorophores that emit in the green region.

In some embodiments of any of the aspects, the quencher molecule is an Eclipse® Dark Quencher. The absorbance maximum for the Eclipse Quencher is at 522 nm, compared to 479 nm for Dabcyl. In addition, the structure of the Eclipse Quencher is substantially more electron deficient than that of Dabcyl and this leads to better quenching over a wider range of dyes, especially those with emission maxima at longer wavelengths (red shifted) such as

Redmond Red and Cyanine 5. In addition, with an absorption range from 390 nm to 625 nm, the Eclipse Quencher is capable of effective quenching of a wide range of fluorophores.

In some embodiments of any of the aspects, the quencher molecule is a QSY® quencher. Non-limiting examples of QSY quenchers include QSY35 (410-500 nm, 475 nm max abs), QSY7 (500-600 nm, 560 nm max abs), QSY21 (590-720nm, 661 nm abs max), and QSY9 (500-600 nm, 562 nm abs max).

In some embodiments of any of the aspects, the quencher molecule is a Qxl™ quencher. Qxl™ quenchers span the full visible spectrum. Non-limiting examples of QXL quenchers include QXL490 (495 nm abs max, can be used as a quencher for EDANS, AMCA, and most coumarin fluorophores), QXL520 (˜ 520 nm abs max, can be used as a quencher for FAM), QXL570 (578 nm abs max, can be used as a quencher for rhodamines (such as TAMRA, sulforhodamine B, ROX) and Cy3 fluorophores), QXL610 (˜610 nm abs max, can be used as a quencher for ROX), and QXL670 (668 nm abs max, can be used as a quencher for Cy5 and Cy5-like fluorophores such as HiLyte™ Fluor 647).

In some embodiments of any of the aspects, the quencher molecule is IRDye QC-1. IRDye QC-1 quenches dyes from the visible to the near-infrared range (500-900 nm, max abs 737 nm).

It is noted that the quencher molecule does not have to be a specifically designed quencher modification. For example, a G base will typically quench nearby fluorescence signals. Accordingly, in some embodiments of any one of the aspects, the quencher is a modified or canonical nucleic acid base capable of inhibiting a fluorescence signal.

### Nucleic Acid Modifications

The various nucleic strands described herein, e.g., the target binding strand, the reporter strand, the quencher strand and/or the reference strand can independently comprise one or more nucleic acid modifications known in the art. For example, the target binding strand, the reporter strand, the quencher strand and/or the reference strand can independently comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs can be modified at the ribose, phosphate, and/or base moiety.

Exemplary nucleic acid modifications include, but are not limited to, nucleobase modifications, sugar modifications, inter-sugar linkage modifications, conjugates (e.g., ligands), and combinations thereof. Nucleic acid modifications are known in the art, see, e.g., US20160367702; US20190060458; U.S. Pat. Nos. 8,710,200; and 7,423,142, contents of all of which are incorporated herein by reference in their entireties.

Exemplary modified nucleobases include, but are not limited to, inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl) uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl) uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2—N(CH3)—N(CH3)—), amide-3 (3′—CH2—C(═O)—N(H)-5′) and amide-4 (3′—CH2—N(H)—C(—O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH2—O—5′), formacetal (3′—O—CH2—O—5′), oxime, methyleneimino, methykenecarbonylamino, methylenemethylimino (MMI, 3′—CH2—N(CH3)—O—5′), methylenehydrazo, methylenedimethylhydrazo, methyleneoxymethylimino, ethers (C3′—O—C5′), thioethers (C3′-S-C5′), thioacetamido (C3′-N (H)—C(═O)—CH2-S-C5′, C3′—O—P(O)—O—SS-C5′, C3′—CH2-NH-NH-C5′, 3′-NHP (O)(OCH3)—O—5′ and 3′-NHP(O)(OCH3)—O-5′.

In some embodiments of any of the aspects, 2′-modified nucleoside comprises a modification selected from the group consisting of 2′-halo (e.g., 2′-fluoro), 2′-alkoxy (e.g., 2′-Omethyl, 2′-Omethylmethoxy and 2′-Omethylethoxy), 2′-aryloxy, 2′—O—amine or 2′—O—alkylamine (amine NH2; alkylamino, dialkylamino, heterocyclyl, arylamino, diaryl amino, heteroaryl amino, dihet.eroaryl amino, ethylene diamine or polyamino), O—CH₂CH₂(NCH₂CH₂NMe₂)₂, methyleneoxy (4′—CH2—O—2′) LNA, ethyleneoxy (4′-(CH₂)₂—O—2′) ENA, 2′-amino (e.g. 2′-NH2, 2′-alkylamino, 2′-dialkylamino, 2′-heterocyclylamino, 2′-arylamino, 2′-diaryl amino, 2′-heteroaryl amino, 2′-diheteroaryl amino, and 2′-amino acid); NH(CH₂CH₂NH)ₙCH₂CH₂-AMINE (AMINE=NH₂, alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), 2′-cyano, 2′-mercapto, 2′-alkyl-thio-alkyl, 2′-thioalkoxy, 2′-thioalkyl, 2′-alkyl, 2′-cycloalkyl, 2′-aryl, 2′-alkenyl and 2′-alkynyl.

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

When present, the nucleic acid modification can be present any wherein the strand comprising the modification. For example, when the target binding strand comprises a nucleic acid modification, such modification can be present in the target binding nucleic acid, in the first hybridization domain, and/or the reference domain of the target binding strand. Alternatively, a nucleic acid modification in the target binding strand can be located outside of the domains of the target binding strand. Similarly, when the reporter strand comprises a nucleic acid modification, such modification can be present in first hybridization domain, in the second hybridization domain or outside the first and second domain of the reporter strand. Likewise, when the quencher strand comprises a nucleic acid modification, such modification can be present in first hybridization domain or outside the first hybridization domain of the quencher strand. In the same way, when the reference strand comprises a nucleic acid modification, such modification can be present in first hybridization domain or outside the first hybridization domain of the reference strand.

The nucleic acid strands described herein, e.g., the target binding strand, the reporter strand, the quencher strand and the reference strand can independently comprise at least one thermally destabilizing or thermally stabilizing modification to optimization of denaturation of a hybridization domain from its complementary domain within a certain thermal channel.

Accordingly, in some embodiments, at least one of the second hybridization domain of the reporter strand and the first hybridization domain of the quencher strand comprises at least one thermally destabilizing modification.

In some embodiments of any one of the aspects described herein, at least one of the first hybridization domain of the reporter strand and the first hybridization domain of the target binding strand comprises at least one thermally destabilizing modification.

In some embodiments of any one of the aspects described herein, the target binding agent is a nucleic acid and comprises at least one thermally destabilizing modification.

In some embodiments of any one of the aspects described herein, at least one of the second hybridization domain of the target binding strand and the first hybridization domain of the reference strand comprises at least one thermally destabilizing modification.

In some embodiments, at least one of the second hybridization domain of the reporter strand and the first hybridization domain of the quencher strand comprises at least one thermally stabilizing modification.

In some embodiments of any one of the aspects described herein, at least one of the first hybridization domain of the reporter strand and the first hybridization domain of the target binding strand comprises at least one thermally stabilizing modification.

In some embodiments of any one of the aspects described herein, the target binding agent is a nucleic acid and comprises at least one thermally stabilizing modification.

In some embodiments of any one of the aspects described berein, at least one of the second hybridization domain of the target binding strand and the first hybridization domain of the reference strand comprises at least one thermally stabilizing modification.

In some embodiments of the various aspects described herein, at least one of the second hybridization domain of the reporter strand and the first hybridization domain of the quencher strand comprises at least one thermally destabilizing modification, and at least one of the first hybridization domain of the reporter strand and the first hybridization domain of the target binding strand comprises at least one thermally stabilizing modification.

In some embodiments of the various aspects described herein, at least one of the second hybridization domain of the reporter strand and the first hybridization domain of the quencher strand comprises at least one thermally destabilizing modification, and the target binding domain of the target binding strand comprises at least one thermally stabilizing modification.

### Target Molecules

The methods, probe sets and/or kits described herein can be used for detecting any desired molecule. For example, any target of interest can be detected using the methods, probe sets and/or kits described herein. Exemplary target molecules that can be detected with the methods, probe sets and/or kits described herein include, but are not limited to, nucleic acids (e.g, DNA, RNA, microRNAs), proteins, saccharides (eg., polysaccharides), lipids, small molecules, and antigens. In some embodiments, the target molecule is a biomolecule. As used herein, a “biomolecule” is any molecule that is produced by a living organisn. including large macromolecules such as nucleic acids (e.g., DNA and RNA such as mRNA), proteins, e.g., antibodies, polysaccharides, lipids and as well as small molecules such as primary metabolites, secondary metabolites, and natural products. The examples included in the disclosure depicting detection of a nucleic acids are for the purpose of illustration and are not intended to limit the scope of the invention.

In some embodiments of any one of the aspects, the target molecule is a nucleic acid.

In some embodiments, target is a nucleic acid such as, for example, nucleic acids of a cellular environment. For example, target is a nucleic acid such as DNA or RNA. In some embodiments the target can be a genomic DNA, cDNA, mRNA, the DNA product of RNA subjected to reverse transcription. In some embodiments, the target is a nucleic acid amplification product.

In some embodiments of any one of the aspects, the target molecule is a protein.

In some embodiments, a target can be a protein target such as, for example, proteins of a cellular environment (e.g., intracellular or membrane proteins). Examples of proteins include, without limitation, fibrous proteins such as cytoskeletal proteins (e.g., actin, arp2/3, coronin, dystrophin, FtsZ, keratin, myosin, nebulin, spectrin, tau, titin, tropomyosin, tubulin and collagen) and extracellular matrix proteins (e.g., collagen, elastin, f-spondin, pikachurin, and fibronectin); globular proteins such as plasma proteins (e.g., serum amyloid P component and serum albumin), coagulation factors (e.g., complement proteins, C1-inhibitor and C3-convertase, Factor VIII, Factor XIII, fibrin, Protein C, Protein S, Protein Z, Protein Z-related protease inhibitor, thrombin, Von Willebrand Factor) and acute phase proteins such as C-reactive protein; hemoproteins; cell adhesion proteins (e.g., cadherin, ependymin, integrin, Ncam and selectin); transmembrane transport proteins (e.g., CFTR, glycophorin D and scramblase) such as ion channels (e.g., ligand-gated ion channels such nicotinic acetylcholine receptors and GABAa receptors, and voltage-gated ion channels such as potassium, calcium and sodium channels), synport/antiport proteins (e.g., glucose transporter); hormones and growth factors (e.g., epidermal growth factor (EGF), fibroblast growth factor (FGF), vascular endothelial growth factor (VEGF), peptide hormones such as insulin, insulin-like growth factor and oxytocin, and steroid hormones such as androgens, estrogens and progesterones); receptors such as transmembrane receptors (e.g., G-protein-coupled receptor, rhodopsin) and intracellular receptors (e.g., estrogen receptor); DNA-binding proteins (e.g., histones, protamines, CI protein); transcription regulators (e.g., c-myc, FOXP2, FOXP3, MyoD and P53); immune system proteins (e.g., immunoglobulins, major histocompatibility antigens and T cell receptors); nutrient storage/transport proteins (e.g., ferritin); chaperone proteins; and enzymes.

In some embodiments of any one of the aspects, the target molecule is in a cell

### Sample/Specimen

Described herein are methods, probe sets and kits for detection of a target in a sample. The term “sample” or “test sample” as used herein can denote a sample taken or isolated from a biological organism, e.g., a subject in need of testing. Exemplary biological samples include tissue samples, such as liver, spleen, kidney, lung, intestine, thymus, colon, tonsil, testis, skin, brain, heart, muscle, and pancreas tissue. Other exemplary biological samples include, but are not limited to, biopsies, bone marrow samples, organ samples, skin fragments and organisms. Materials obtained from clinical or forensic settings are also within the intended meaning of the term biological sample. In one embodiment, the sample is derived from a human, animal or plant. In one embodiment, the biological sample is a tissue sample, preferably an organ tissue sample. In one embodiment, samples are human. The sample can be obtained, for example, from autopsy, biopsy, muscle punch, or from surgery. It can be a solid tissue or solid tumor such as parenchyme, connective or fatty tissue, heart or skeletal muscle, smooth muscle, skin, brain, nerve, kidney, liver, spleen, breast, carcinoma (e.g., bowel, nasopharynx, breast, lung, stomach etc.), cartilage, lymphoma, meningioma, placenta, prostate, thymus, tonsil, umbilical cord or uterus. The tissue can be a tumor (benign or malignant), cancerous or precancerous tissue. The sample can be obtained from an animal or human subject affected by disease or other pathology or suspected of same (normal or diseased), or considered normal or healthy.

In some embodiments of any of the aspects, the biological sample is a sputum sample, a pharyngeal sample, or a nasal sample. In some embodiments of any of the aspects, the biological sample is cells, or tissue, or peripheral blood, or bodily fluid. In some embodiments, the biological sample is a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; interstitial fluid; or tissue sample. The term also includes a mixture of the above-mentioned samples. The term “test sample” also includes untreated or pretreated (or pre-processed) biological samples.

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. The skilled artisan is well aware of methods and processes appropriate for pre-processing of biological samples required for detection of targets, such as nucleic acids as described herein.

### Generating the Probes

The nucleic acid strands described herein can be synthesized using any probe or oligonucleotide synthesis methods known in the art including, but not limited to, solid phase oligonucleotide synthesis, RNA synthesis, or through the use of an automated DNA/RNA synthesizer (e.g., Sierra Biosystems (Sonora, CA), Biolytic (Fremont, CA)). Alternatively, the probes can be synthesized by one or more commercial sources including, but not limited to, Bio Basic (Amherst, New York), Integrated DNA technologies (Coralville, lowa), Trilink Biotechnologies (San Diego, CA) and the like.

### Kits

In one aspect, provided herein is a kit for detecting a target molecule. The kit can comprise any of one of the probe sets described herein and packaging and materials for use therefore.

### Applications

The methods, detection probe sets and kits described herein can be used with any method for detecting a target molecule, e.g., a gene sequence or RNA species in a single-cell or tissue known in the art. Non-limiting examples of applications that can be used with the nucleic acid encoded probes and compositions described herein.

In one embodiment, the detection probe set described herein can be used for transcription profiling in single cells In some embodiments, a transcriptional state of a cell is imaged by detecting and distinguishing individual mRNAs. Florescence In-Situ Hybridization (FISH) allows single mRNAs molecule in fixed cells to be labeled and imaged. This is accomplished by hybridizing the mRNA with a nucleic acid encoded probe having a sequence complementary to the mRNA sequence.

In another embodiment, the nucleic acid encoded probes described herein can be used in methods for mapping chromosome structures. Chromosomal rearrangements have been implicated in many forms of cancer, and recent investigations revealed that chromosomes in eukaryotes are packed in a non-linear and complex fashion. The nucleic acid encoded probes can be applied to image the structure of chromosomes and determine their conformation in single cells. A physical image of the chromosomes in cells with the addresses of individual genes will allow for an unprecedented look at how the genome is compacted, compare organization in transcriptionally active versus repressed regions, and detect subtle changes in genomic structure in tumor cells.

In another embodiment, the nucleic acid encoded probes can be used in methods for imaging transcription factor binding in single cells. Transcription factors (TFs) control genes in transcriptional networks through binding sites on the DNA and interactions with regulatory proteins. The distribution of positions and binding states of a particular TF on the chromosome determines the transcriptional program it is accessing in the cell. By incorporating a transcription factor binding domain into the nucleic acid encoded probe, the physical location of individual TEs can be determined One of skill in the art will understand that modified nucleosides that achieve a higher melting point for the DNA binding domain, or render the interaction irreversible may be necessary to permit temperature changes to thermal channels that permit denaturation of e.g., at least one fluorophore/quencher pair to permit detection of the transcription factor.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

Some embodiments of the technology described herein can be defined according to any of the following numbered paragraphs:

Paragraph 1: A method for detecting a target molecule in a sample, the method comprising: (a) providing a detection probe set, wherein detection probe set comprises: (i) a first nucleic acid strand comprising a target binding domain linked to a first hybridization domain; (ii) a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and (iii) a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and wherein a melting temperature of the third strand hybridizing with the second strand is lower than a melting temperature of the second strand hybridizing with the first strand, and wherein a wherein a melting temperature of the third strand hybridizing with the second strand is lower than a melting temperature of the first strand binding with the target molecule; and (b) analyzing the binding of the probe-set to the target by assessing a detectable signal produced as a function of temperature.

Paragraph 2. A method for multiplex detecting of target molecules in a sample, the method comprising: (a) providing a plurality of detection probe sets, wherein each detection probe set comprises: (i) a first nucleic acid strand comprising a target binding domain linked to a first hybridization domain; (ii) a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and (iii) a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and wherein a melting temperature of the third strand hybridizing with the second strand is lower than a melting temperature of the second strand hybridizing with the first strand, and wherein a wherein a melting temperature of the third strand hybridizing with the second strand is lower than a melting temperature of the first strand binding with the target molecule, and wherein the melting temperature of the third strand hybridizing with the second strand in at least one probe set is different from the melting temperature of the third strand hybridizing with the second strand in at least one other probe set; and (b) analyzing the binding of the probe-sets to the targets by assessing a detectable signal produced as a function of temperature.

Paragraph 3: The method of paragraph 2, wherein the melting temperature of the third strand hybridizing with the second strand in at least one probe set is at least 5° C. lower than the melting temperature of the third strand hybridizing with the second strand in at least one other probe set.

Paragraph 4: The method of any one of paragraphs 2-3, wherein reporter molecules in the different probe sets are the same.

Paragraph 5: The method of any one of paragraphs 2-3, wherein the reporter molecule in at least one probe set is different from the reporter molecule in at least one other probe set.

Paragraph 6: The method of any one of paragraphs 1-5, wherein the melting temperature of the third strand hybridizing with the second strand is at least 5° C. lower than the melting temperature of the second strand hybridizing with the first strand.

Paragraph 7. The method of any one of paragraphs 1-6, wherein the melting temperature of the third strand hybridizing with the second strand is at least 5° C. lower than the melting temperature of the first strand binding with the first strand.

Paragraph 8: The method of any one of paragraphs 1-7, wherein at least one of the first, second or third strand comprised a nucleic acid modification.

Paragraph 9: The method of any one of paragraphs 1-8, wherein the first hybridization domain of the first strand and/or the second hybridization domain of the second strand comprises a duplex stabilizing modification.

Paragraph 10: The method of any one of paragraphs 1-9, wherein the third hybridization domain of the second strand and/or the fourth hybridization domain comprises a duplex destabilizing modification.

Paragraph 11: The method of any one of paragraphs 1-10, wherein the reporter molecule and the quencher molecule are a FRET pair.

Paragraph 12: The method of any one of paragraphs 1-11, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

Paragraph 13. The method of any one of paragraphs 1-12, wherein the quencher molecule is a dark quencher.

Paragraph 14: The method of any one of paragraphs 1-13, wherein the first strand further comprises a reporter domain linked to the target binding domain.

Paragraph 15: The method of paragraphs 14, wherein the probe set further comprises a fourth nucleic acid strand, wherein the fourth nucleic acid strand comprises a fifth hybridization domain, wherein the fifth hybridization domain comprises a nucleotide sequence substantially complementary to a nucleotide sequence of the reporter domain of the first strand, and optionally, the fourth strand comprises a reporter molecule.

Paragraph 16: The method of any one of paragraphs 1-15, wherein said analyzing the binding of the probe-set to the target comprises detecting the detectable signal at a first temperature and at a second temperature, wherein one of the first or second temperature is lower than the melting temperature of the third strand hybridizing with the second strand and the other of the first or second temperature is higher than the melting temperature of the third strand hybridizing with the second strand.

Paragraph 17: A probe set for detecting a target molecule, the probe set comprising: (i) a first nucleic acid strand comprising a target binding domain linked to a first hybridization domain; (ii) a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and (iii) a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon.

Paragraph 18: A probe set for multiplex detection of target molecule, comprising a plurality of detection probe sets, wherein each detection probe set comprises: (i) a first nucleic acid strand comprising a target binding domain linked to a first hybridization domain; (ii) a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and (iii) a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and wherein a melting temperature of the third strand hybridizing with the second strand is lower than a melting temperature of the second strand hybridizing with the first strand, and wherein a wherein a melting temperature of the third strand hybridizing with the second strand is lower than a melting temperature of the first strand binding with the target molecule, and wherein the melting temperature of the third strand hybridizing with the second strand in at least one probe set is different from the melting temperature of the third strand hybridizing with the second strand in at least one other probe set.

Paragraph 19: The probe set of paragraph 18, wherein the melting temperature of the third strand hybridizing with the second strand in at least one probe set is at least 5° C. lower than the melting temperature of the third strand hybridizing with the second strand in at least one other probe set.

Paragraph 20: The probe set of any one of paragraphs 18-19, wherein reporter molecules in the different probe sets are the same.

Paragraph 21: The probe set of any one of paragraphs 18-19, wherein the reporter molecule in at least one probe set is different from the reporter molecule in at least one other probe set.

Paragraph 22. The probe set of any one of paragraphs 17-21, wherein the melting temperature of the third strand hybridizing with the second strand is at least 5° C. lower than the melting temperature of the second strand hybridizing with the first strand.

Paragraph 23: The probe set of any one of paragraphs 17-22, wherein the melting temperature of the third strand hybridizing with the second strand is at least 5° C. lower than the melting temperature of the first strand binding with the first strand.

Paragraph 24: The probe set of any one of paragraphs 17-23, wherein at least one of the first, second or third strand comprised a nucleic acid modification.

Paragraph 25: The probe set of any one of paragraphs 17-24, wherein the first hybridization domain of the first strand and/or the second hybridization domain of the second strand comprises a duplex stabilizing modification.

Paragraph 26: The probe set of any one of paragraphs 17-25, wherein the third hybridization domain of the second strand and/or the fourth hybridization domain comprises a duplex destabilizing modification.

Paragraph 27. The probe set of any one of paragraphs 17-26, wherein the reporter molecule and the quencher molecule are a FRET pair.

Paragraph 28 The probe set of any one of paragraphs 17-27, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

Paragraph 29: The probe set of any one of paragraphs 17-28, wherein the quencher molecule is a dark quencher.

Paragraph 30: The probe set of any one of paragraphs 17-29, wherein the first strand further comprises a reporter domain linked to the target binding domain.

Paragraph 31. The probe set of any one of paragraphs 17-30, wherein the probe set further comprises a fourth nucleic acid strand, wherein the fourth nucleic acid strand comprises a fifth hybridization domain, wherein the fifth hybridization domain comprises a nucleotide sequence substantially complementary to a nucleotide sequence of the reporter domain of the first strand, and optionally, the fourth strand comprises a reporter molecule.

Paragraph 32. A kit for detecting a target molecule, the kit comprising a probe-set of any one of paragraphs 17-31.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M J. Gait, ed., 1984); “Animal Cell Culture” (R. I. Freshney, ed., 1987), “Methods in Enzymology” (Academic Press, Inc.); “Current Protocols in Molecular Biology” (F. M. Ausubel et al., eds., 1987, and periodic updates); “PCR: The Polymerase Chain Reaction”. (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “hybridize” refers to the interaction or annealing of two single-stranded nucleic acids into a double-stranded nucleic acid (e.g., duplex) under specific hybridization conditions. Typically, two single-stranded nucleic acids form a duplex when they have sufficient complementarity to each other under a give set of conditions. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides

As used herein, the term “thermal channel” refers to a distinct range of temperatures at which a given double-stranded labeled region(s) of the probe(s) denature, while the other double-stranded labeled region(s), including the double-stranded region formed by the probe binding to the target nucleic acid, remain annealed. For example, at a particular thermal channel, a double-stranded labeled region denatures, releasing the e.g., quenched fluorophore and permitting detection of the target in a cell or tissue. Where the term “thermal channel” is used to describe the double-stranded region formed by the probe binding to the target nucleic acid, the temperature range is that which will denature the probe from the target nucleic acid sequence In some embodiments, the temperature channel comprises a range of temperatures within 5° C. (e.g., 50° C.-55° C.), within 10° C. (e.g., 50° C.-60° C.), within 15° C. (e.g., 50° C.-65°C), within 20° C. (e.g, 50° C.-70° C.), or greater

As used herein, the term “substantially complementary” refers to two nucleic acid strands that are sufficiently complimentary in sequence to anneal and form a stable duplex, provided that the complementarity is sufficient to produce a melting point within the desired range. The complementarity does not need to be perfect; there can be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region. In one embodiment, the single-stranded region of the DNA or RNA encoded probe is 100% complementary to a given region of the target sequence (e.g., comprises no mismatches) in order to permit the generation of a binding region that has a melting point that his higher than the double-stranded region of the probe.

The term “thermally destabilizing modification(s)” includes modification(s) to a nucleic acid sequence such that the sequence has a lower overall melting temperature (Tm) (preferably a Im with one, two, three or four degrees lower than the Im of the nucleic acid sequence without having such modification(s). Exemplary thermally destabilizing modifications can include, but are not limited to, abasic modifications, mismatches with the opposing nucleotide in the opposing strand (e.g., G:G, G:A, G:U, G:T, A:A, A:C, C:C, C:U, C:T, U:U, T:T and U:T), acyclic nucleotides (e.g, unlocked nucleic acids (UNA) and glycol nucleic acid (GNA), α-nucleotides, nucleotides with impaired Watson-Crick hydrogen bonding, nucleotides with non-canonical bases (such as inosine, nebularine, 2-aminopurine, 2,4-diflurotoluene, 5-nitroindole, 3-nitorpyrrole, 4-fluro-6-methylbenzimidazole, and 4-methylbenzimidazole), universal nucleobases with reduced or abolished capability to form hydrogen bonds with the opposing bases, and phosphate modifications (such as phosphorthioates, and alklphosphonates). Exemplary thermally destabilizing modifications are described, for example, in WO 2011/133876 and WO 2010/0011895, contents of both of which are incorporated herein by reference in their entireties.

Conversely, the term “thermally stabilizing modification(s)” includes modification(s) to a nucleic acid sequence such that the sequence has a higher overall melting temperature (Tm) (preferably a Tm with one, two, three or four degrees lower than the Tm of the nucleic acid sequence without having such modification(s). In the context of the methods and compositions described herein, a thermally stabilizing modification can be included in any region of the probe to achieve a higher melting temperature, thereby permitting melting of the probe within a desired thermal channel. Exemplary thermally stabilizing modifications include, but are not limited to 2′-fluoro modifications, locked nucleic acid (LNA) bases, minor groove binders (MGBs), 5-hydroxybutynyl-2′-deoxyuridine (SuperT), 5-Me-pyridines, 2-amino-deoxyadenosine, Trimethoxystilbene, RNA bases, methylated RNA bases, 2′ Fluoro bases, and pyrene.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Multiplex Fluorescent Cellular Imaging with DNA Encoded Thermal Channels and Uses Thereof

Optical fluorescence microscopy is a powerful tool to visualize biomolecules at both the single-cell and tissue level to reveal complex cellular signal networks and disease states. However, the capability of visualizing many biological targets is fundamentally limited by the range of the color palette. Provided herein is an elegant and effective method to expand the color palette by adding thermal channel dimension to the fluorescence spectral imaging. The inventors have engineered DNA thermal probes that only illuminate fluorescent signals at desired temperature channels and remain dark at other temperature channels. Five temperature channels are demonstrated herein that have minimal crosstalk and that can be applied for biological imaging. The temperature channel switching time just needs 5 seconds, which is generally over 100-fold faster than the buffer exchange methods in conventional imaging. Through the combination of thermal and fluorophore channels, the inventors showed 15 colors of RNA imaging in fixed cells. The thermal multiplexed imaging method has wide applications in the spatial biology area.

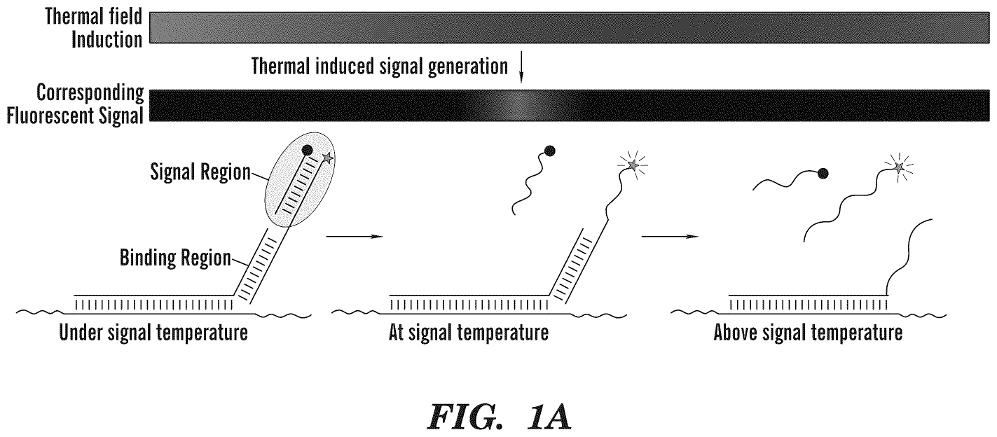

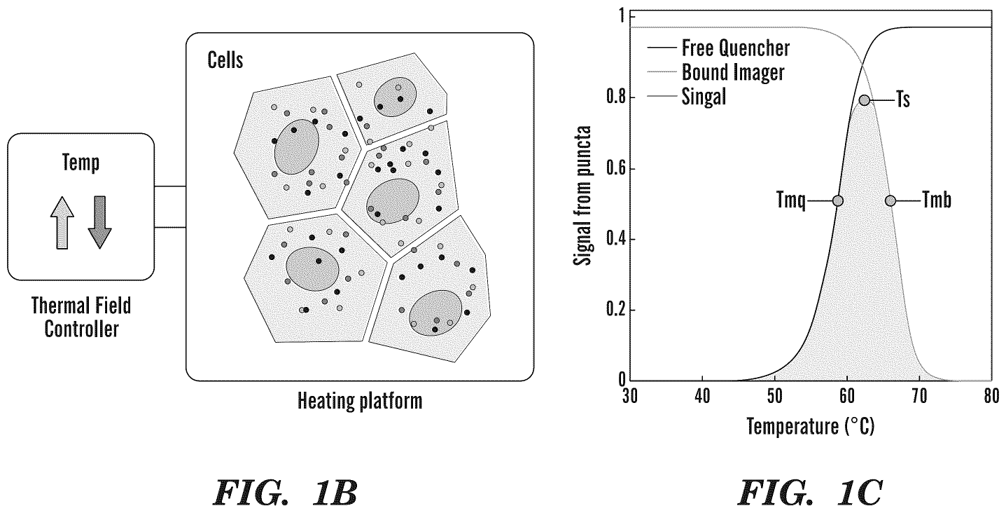

To fundamentally address the color limitation of fluorescence imaging, the inventors developed a DNA thermal scope system that takes advantage of programmable melting of DNA probes in situ by the induction of thermal fields to generate fluorescence signals at pre-determined thermal channels (FIG. 1A). The thermal fluorescent imaging is demonstrated herein to work robustly in fixed Hela cells. The channel switching time only takes 5 seconds, much faster than the typical microfluidic iterative washing system (2 mins) of conventional imaging. The inventors showed five thermal channels with non-observable crosstalk. 15-plex RNA profiling in a single cell is successfully demonstrated in situ by combining the thermal plex with widely used three fluorescent channels. The DNA thermal scope based on DNA encoded thermal channels will be a powerful tool to expand the toolbox for spatial biology.

### Results

Design principles of fluorescent imaging with DNA thermal probes. The core part of the thermal-plex methods is the design of thermal DNA probes that allows efficient signal generation at desired temperature channels and avoids signal crosstalk between different temperature channels. The DNA thermal probe is designed to have two regions, the signal region and the binding region, to achieve these goals. The signal region contains a DNA duplex with a fluorophore and quencher pair labeled at the end. The dye's fluorescence is quenched unless the quencher strand is melted off from the probe by thermal field induction at the desired signal temperature channel. The binding region is designed to bind the target DNA strand, which has already been attached to the target biomolecules in situ (FIG. 1A). The binding region generally has higher thermal stability than the signal region to ensure the signal intensity. After the probe is bound to the DNA labeled on a biological target in situ, the thermal field is applied to induct the signal (FIG. 1B). At the desired signal temperature, the quencher strand is melted off from the probe, and the imager stays with the target biomolecules through the binging region. Consequently, the target biomolecules can be visualized through the fluorescence signal released by the imager strand. The thermal profile of the quencher attached to the puncta is shown in FIG. 1C. When switched to a higher temperature channel, the binding region is also melted off and the fluorescent signal will disappear, which is shown in the brown curve in the FIG. 1C. The thermal profile intersection of quencher region and binding region will give the thermal spectrum of a designed DNA thermal probe, as shown in FIG. 1C. The signal temperature (Ts) is determined at the peak region of the thermal spectrum.

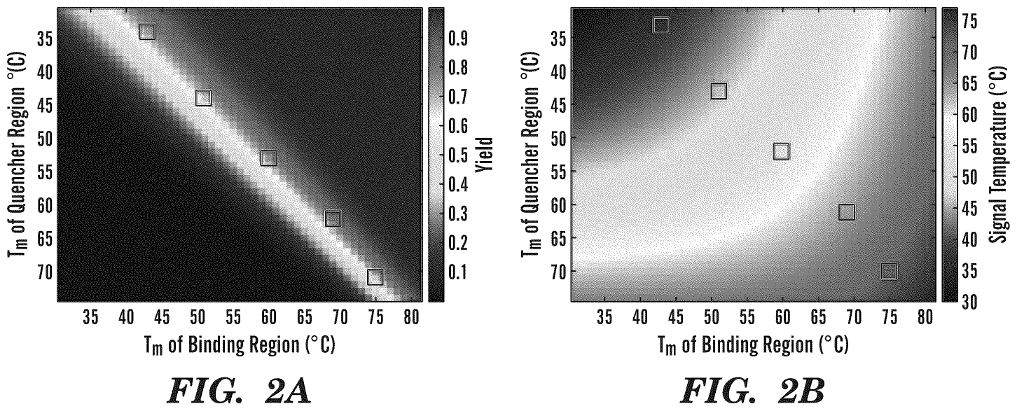

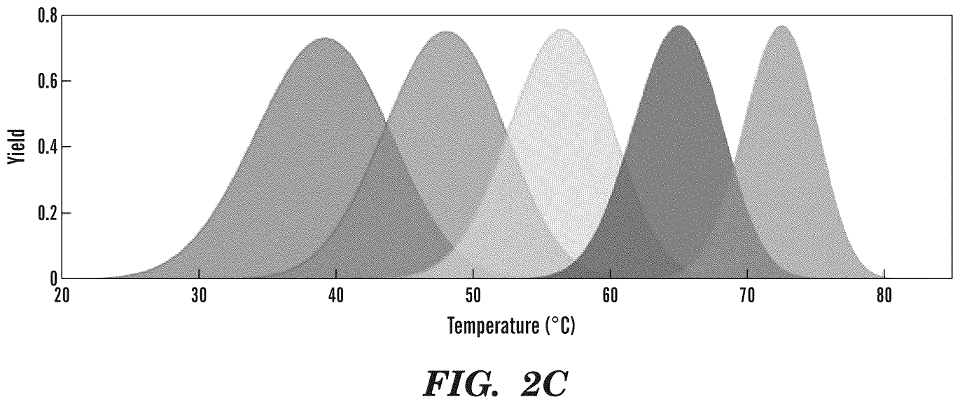

Simulation guided design of thermal channels with DNA thermal probes for multiplexed imaging. To implement the DNA thermal-plex for cellular imaging, the DNA thermal probes need to have two key properties: (1) sufficient efficiency to generate fluorescent signal at its signal temperature, (2) as many as channels that have non-overlapped thermal spectrum for multiplexed imaging. To achieve the two goals, the inventors explored the design space with computational simulation under all the combinations of binding region and quenching's melting temperature, resulted heatmaps of the signal yield and the signal temperature are shown in FIGS. 2A and 2B. As the melting temperature of binding region is higher than the quencher region in the upper-right triangular area, the signal yield is also higher compared to the lower-right triangular area. The design located in the corner of upper-right area gives relatively fat thermal spectrum, resulting small temperature bandwidth for the multiplexed imaging. To select the design for multiplexed imaging, the parameters of the probe should be in the upper-right triangular area but close to the diagonal region of the square space to ensure the relatively high signal yield and higher multiplexed channels. Based on the theoretical analysis, as shown in FIG. 2C, five thermal channels (39° C., 48° C., 57° C., 65° C., and 72° C.) can be created with minimal crosstalk for spatial biological applications.

Validation of in situ RNA imaging with DNA thermal probes: To validate the method and principle described above, a DNA thermal probe is designed to target the APC RNA transcript in Hela cells. The melting temperature of the signal region and binding region is designed to be 53° C. and 61° C., respectively. The resulting optimal signal temperature is about 57° C. The primary probe for the APC RNA is designed with Oligominer4. After the sequence generation of primary probes, a reference probe binding region is appended to the primary APC probes on the 5′ end and the DNA thermal probe binding region is attached to the 3′ end. Both the appended sequences for the reference probe and thermal probe have been checked though blast to avoid any sequence alignment with human transcriptome.5

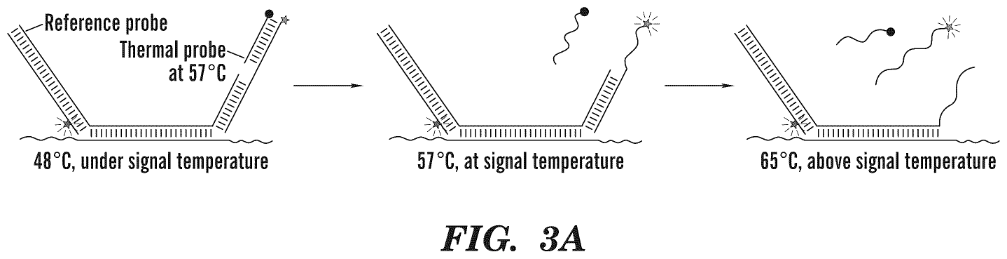

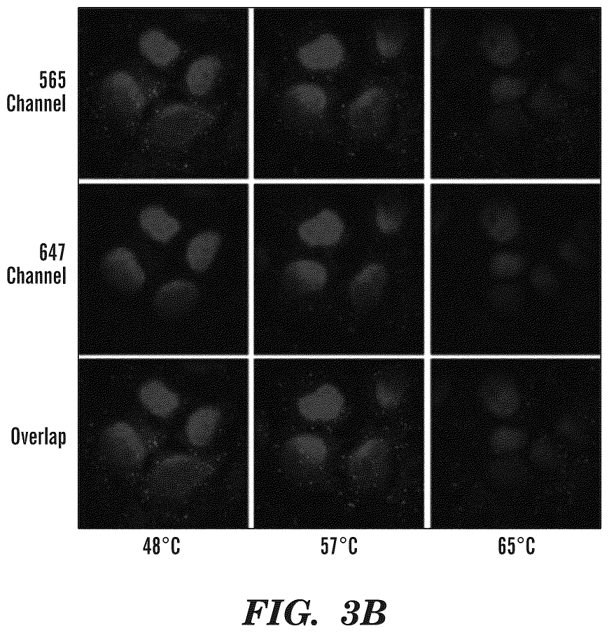

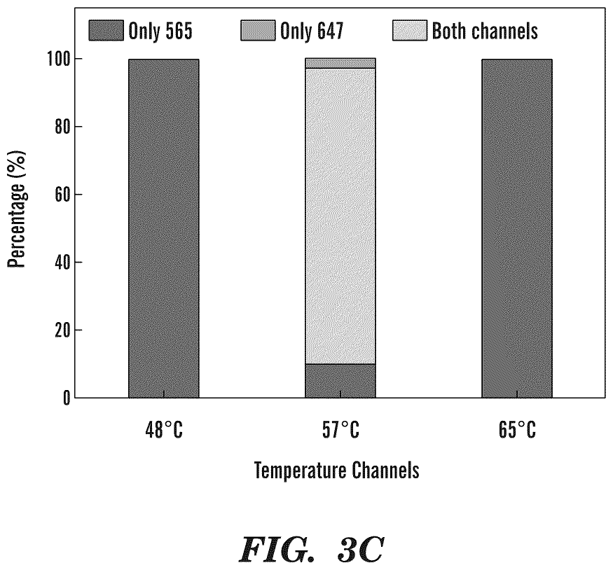

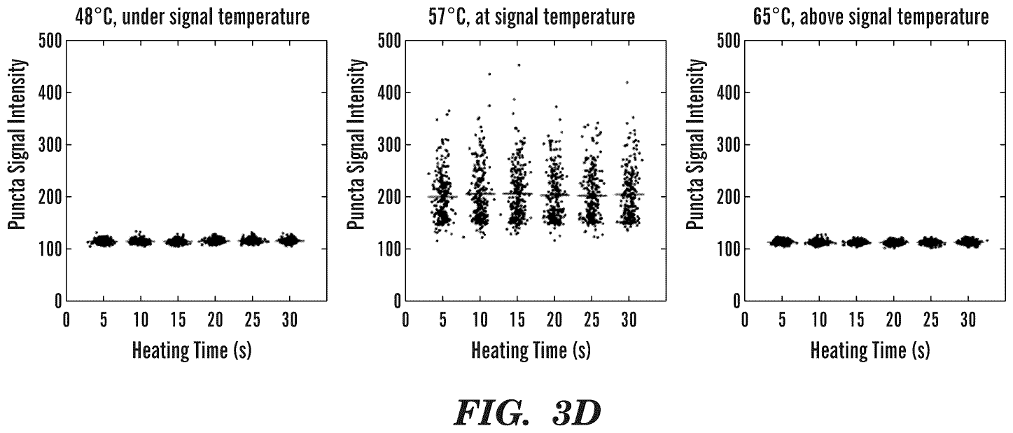

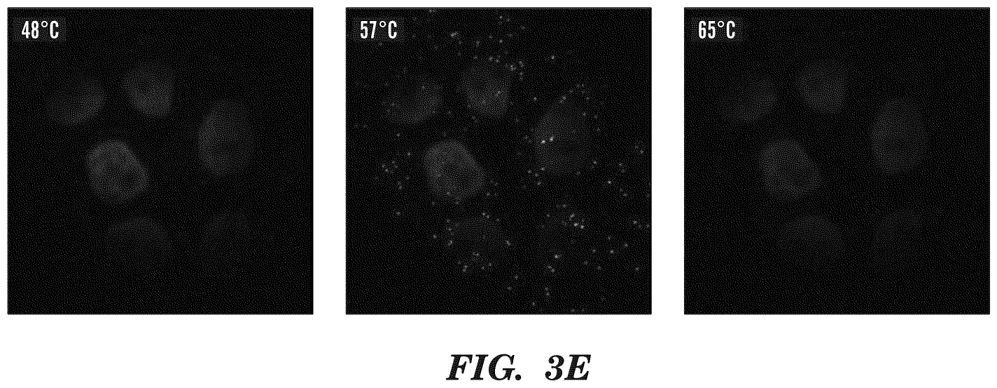

To do the RNA imaging in situ, the Hela cells were cultured and fixed on a substrate with a temperature control module. The designed primary probes were incubated with the fixed cell overnight for sufficient binding with the target RNA, which then followed by the binding process of reference and thermal probes onto the primary probes (FIG. 3A). The reference probes are labeled with Atto565 dye, and the thermal probes is encoded with Alex647 dye. Thermal inductions at a set of temperatures are then applied to the substrate for the fluorescent signal generation by melting the DNA strands in situ. Typical fluorescent imaging steps are applied to read out the signal. As shown in FIG. 3B, at the temperature lower than the signal temperature, such as 48° C., only the 565 nm channel with reference probes show the RNA puncta signal. While at signal temperature, both the 565-nm and 647-nm channel shows the RNA puncta signal. When the temperature switched to 72 degrees, only the 565-nm showed the puncta signal again, indicating that the imager strand was melted off the in situ binding spot. The puncta information has been analyzed at different temperature and fluorophore channels as shown in FIG. 3C, 90% of the puncta's position align very well from two fluorophore channels. Other unalignment puncta may attributed to the unspecific binding of the fluorescent probes. The great consistency of signal across the reference probes and thermal probes demonstrate that thermal probes can precisely illuminate target RNA under designed signal temperature. While at lower or higher temperature channels, the signal is completely removed, thereby preventing signal crosstalk.

Fast dissociation kinetics for the channel switching: As thermal channel colors rely on the dissociation of the quencher strand under the signal temperature, the speed of the color generation is very fast and can be achieved in seconds based on kinetics analysis. To test the temperature channel switching time, fluorescent imaging data is collected after heating the substrate under the temperature lower than the signal temperature, at the signal temperature, and higher than the signal temperature for different lengths of time, as shown in FIGS. 3E and 3F. It is shown that 5 seconds is enough to fully melt off the quencher strand to generate the signal at signal temperature and remove the imager strands above the signal temperatures.

In the iterative binding methods for multiplexed fluorescent imaging, it is the washing steps that are usually time consuming and can require about 30 mins or even hours to remove the previous round of imagers and allow the binding of current imagers. The DNA thermal scope can speed up the imaging process by over 100-fold.

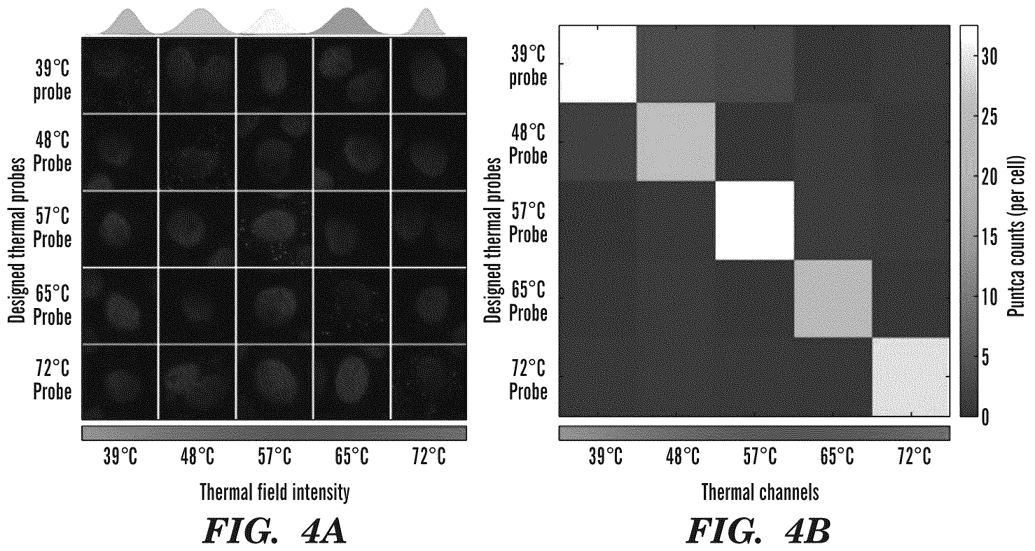

Design of five thermal channels for fluorescence imaging: As the inventors have validated that the DNA thermal probes can release fluorescence signal at designed signal temperature and remain dark at other temperature channels, they then attempted to engineer five thermal channels without signal crosstalk. The designed signal temperatures for the five DNA thermal probes are around 39° C., 48° C., 57° C., 65° C., 72° C. After the binding of five different DNA thermal probes to the APC primary probes, the thermal field is applied to observe the fluorescent signal at the five designed temperature channels. As shown in FIG. 4A, all the samples incubated with designed DNA thermal probes only show the fluorescent RNA puncta signal at the corresponding temperature channel, while remaining dark on the other channels. The inventors also analyzed the number of RNA puncta per cell for each designed thermal probes, and the RNA puncta numbers per cells detected by each thermal probes are in the same level (FIG. 4B).

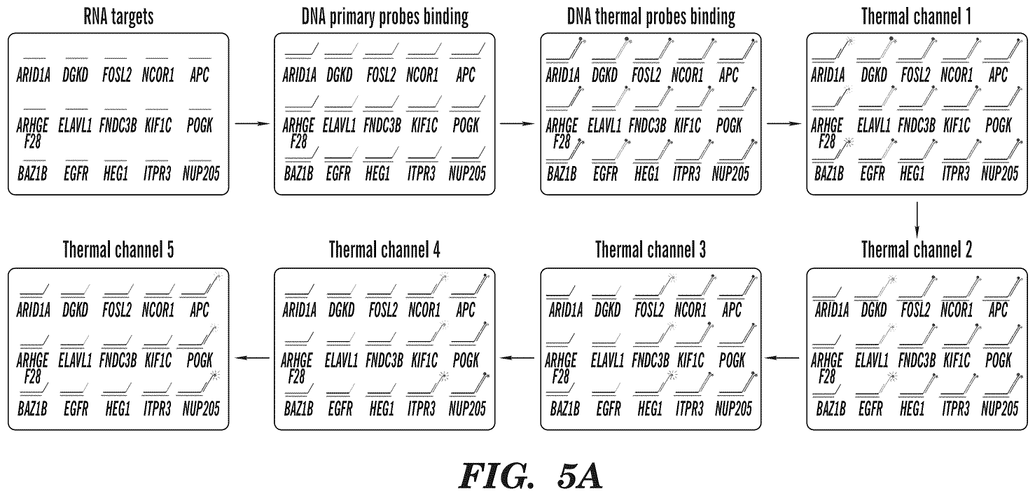

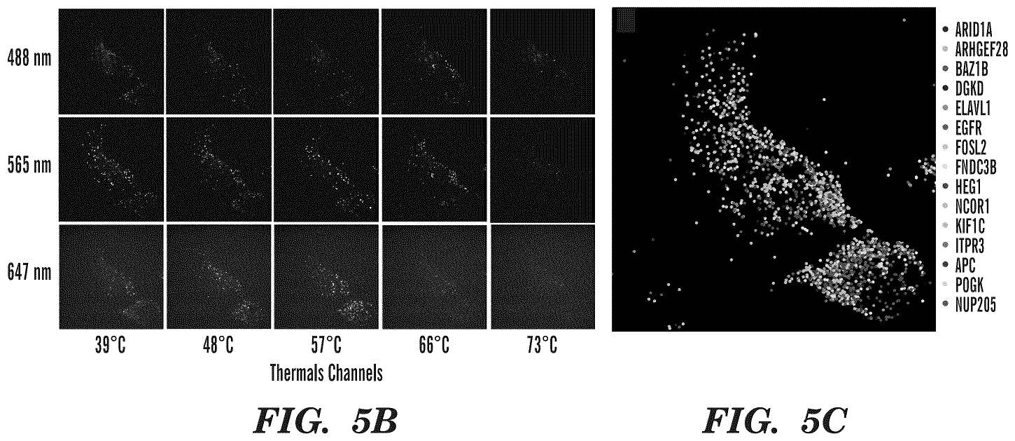

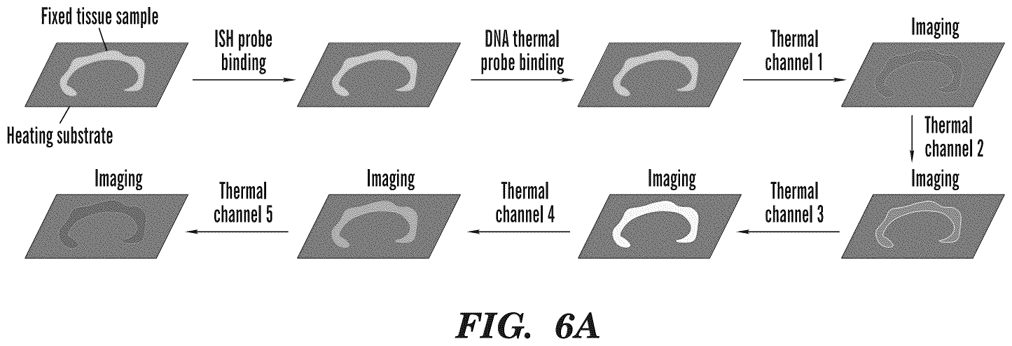

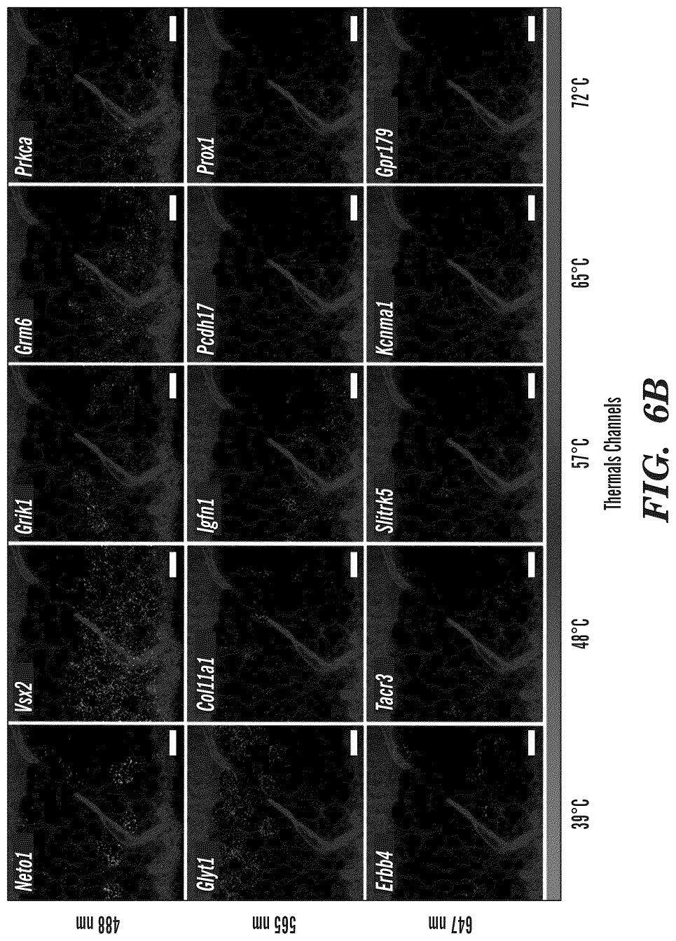

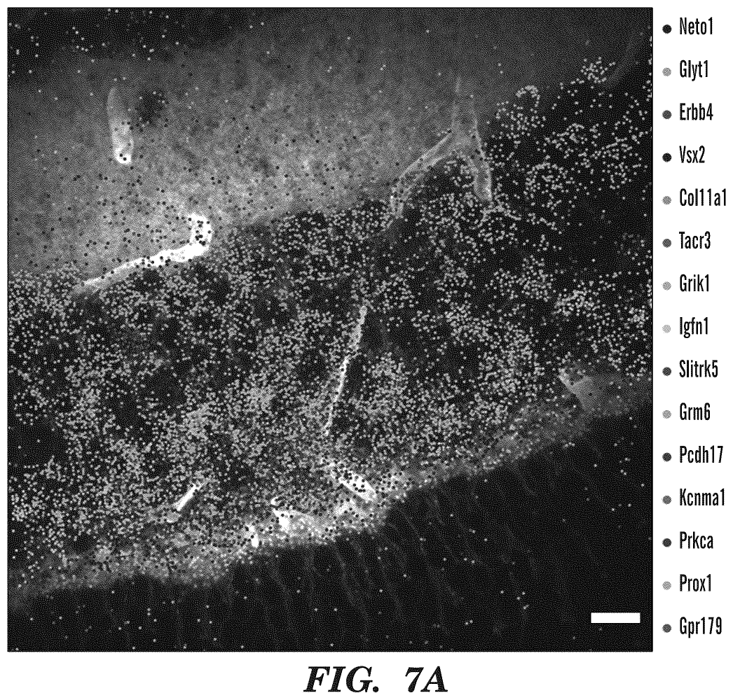

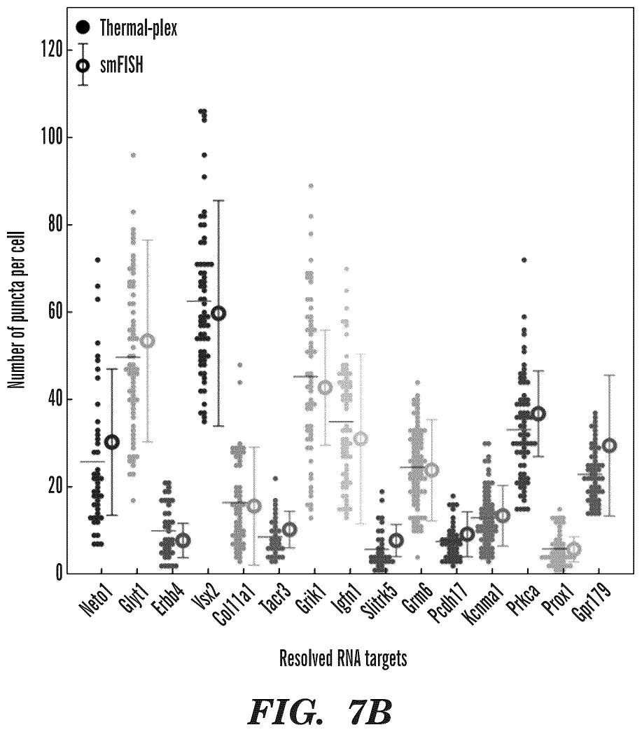

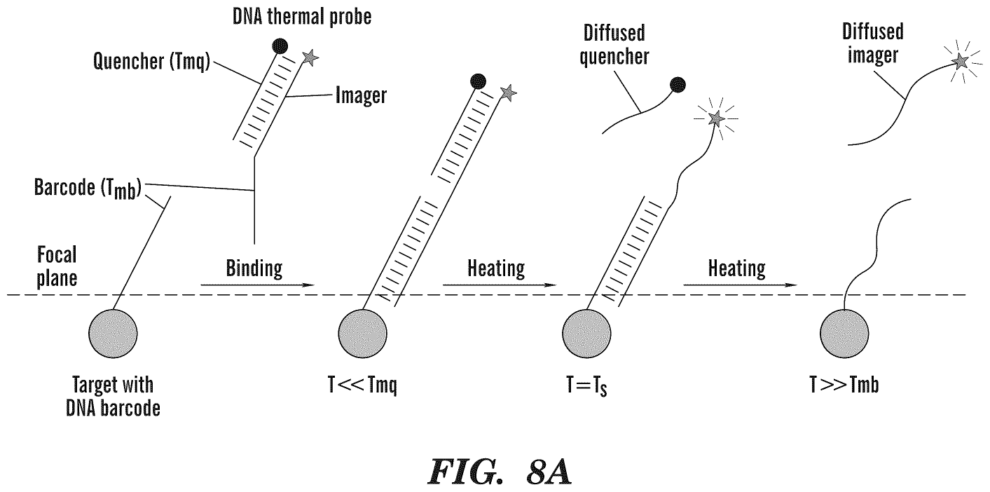

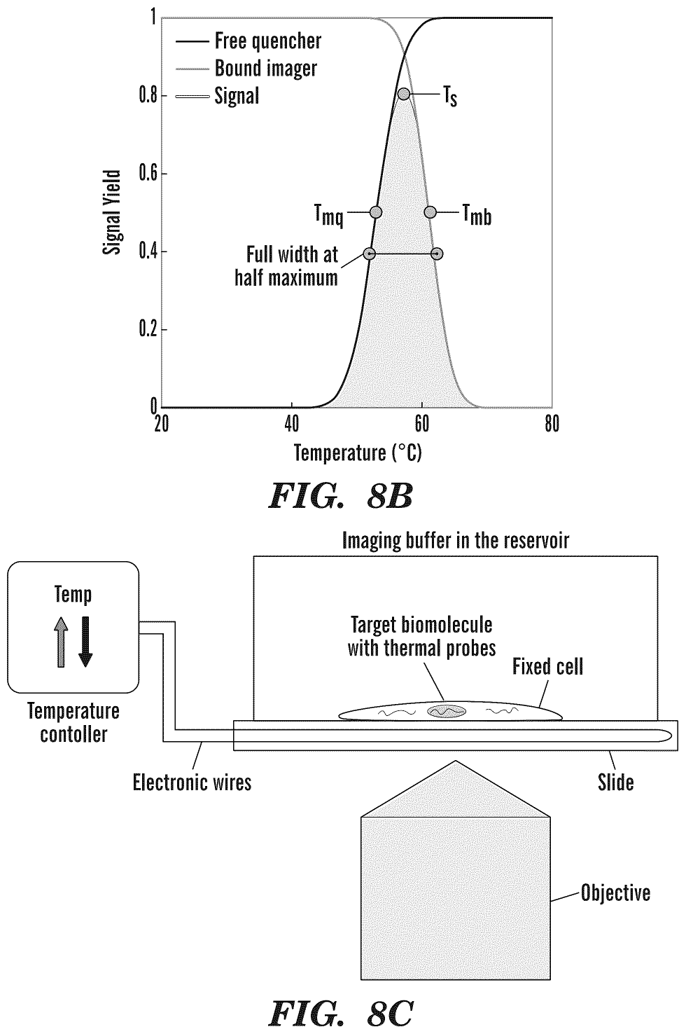

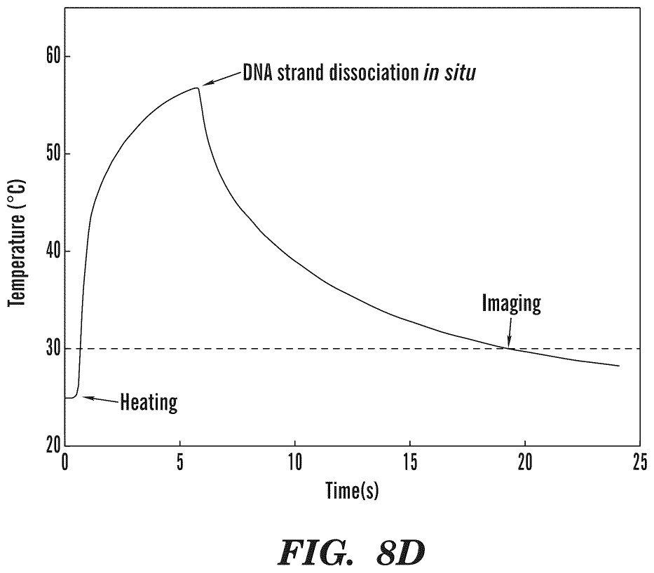

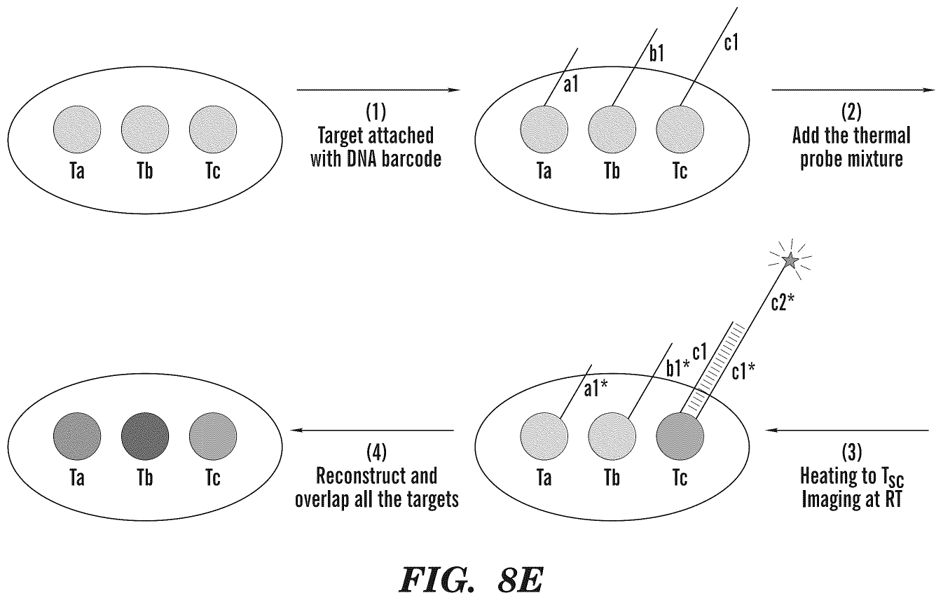

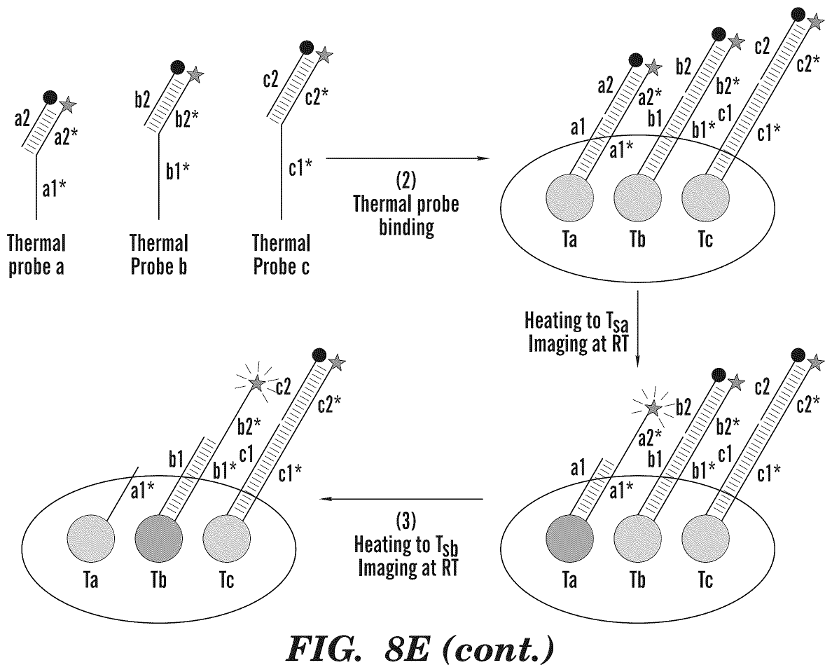

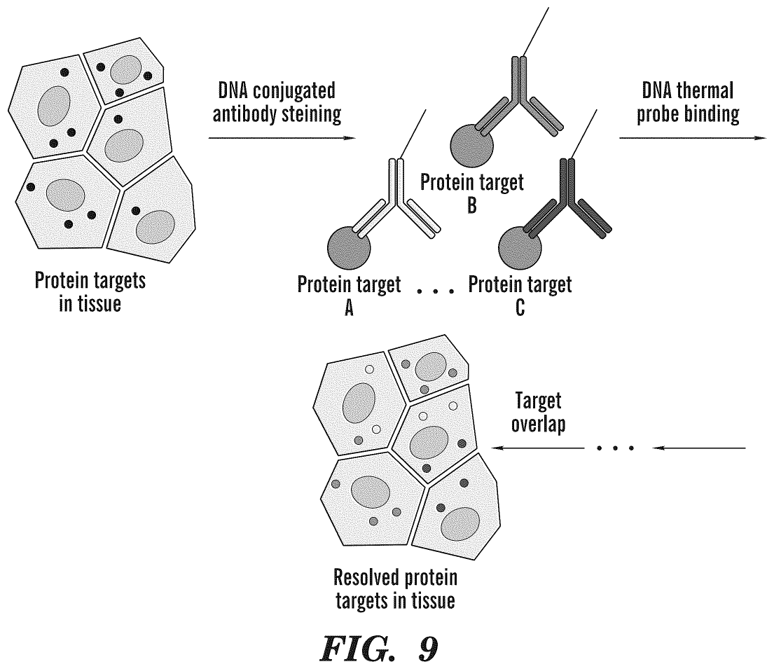

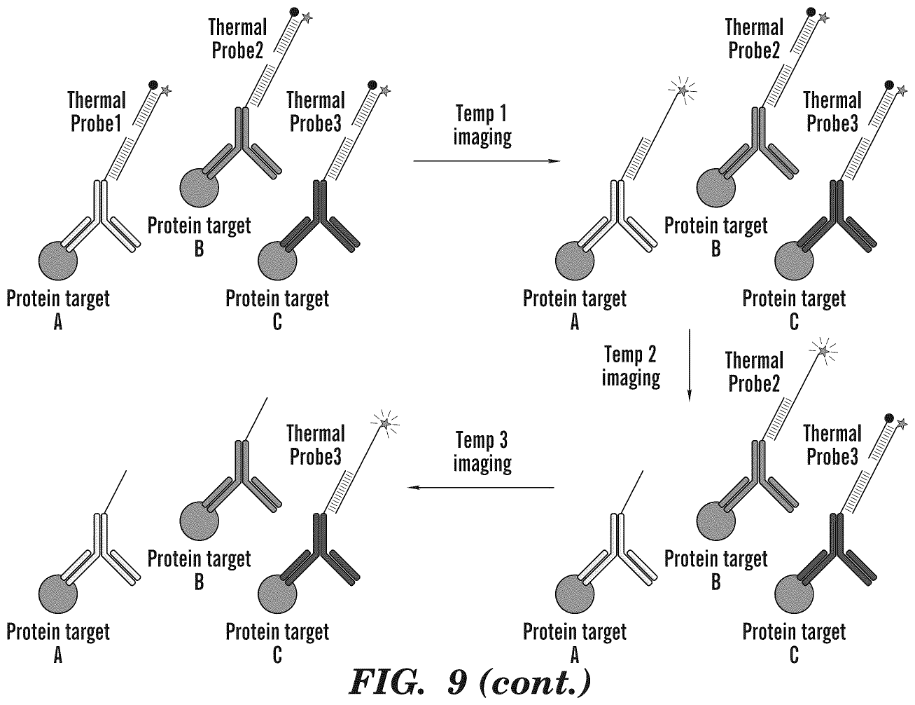

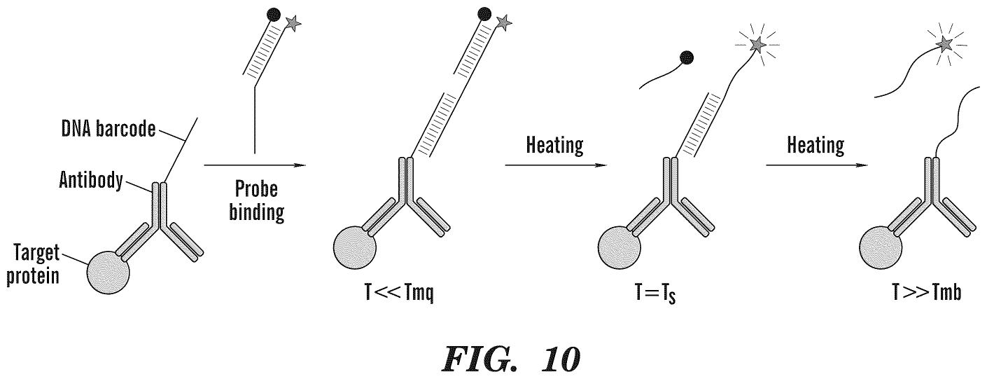

Multiplexed cellular RNA imaging: The thermal-plex methods provided an additional signal channel other than the fluorophore channel, which is powerful to increase bioimaging's multiplex capabilities. The combination of five engineered thermal channels with three fluorophore channels is expected to give the capability to improve the multiplexity of fluorescence imaging from 3 to 15. To demonstrate the multiplexed imaging capability, the inventors designed 15 sets of thermal probes to target 15 RNA targets in Hela cells. Every three thermal probes are encoded with three different fluorophores, Atto-488, Atto-565, and Alex-647, respectively. As shown in the scheme of FIG. 5A, during the imaging process, after each round of temperature channel switching, the image will be taken at 488-nm, 565-nm, and 647-nm channels. The fifteen images for each designed thermal probe are shown in FIG. 5B. The overlapping image for a single cell with fifteen colors is shown in FIG. 5C. The overall time for the imaging time only takes less than 10 mins.

The multiplexed fluorescence imaging method based on thermal-field induced sequential melting provides a convenient and efficient way for monitoring in situ spatial biology. Previous described multiplexed fluorescent imaging methods are achieved by spectrum⁶, geometry⁷, and iterative encoding⁸. Although spectrum encoding is a straightforward and convenient method to perform multiplex imaging, it requires multiple fluorophores, which is fundamentally limited by 3-4 colors to choose. The geometric encoding requires a relatively large size of scaffold to put fluorophores to have enough space for geometric differentiation under diffraction-limited microscope. Iterative binding of imagers is powerful to increase the multiplexity with unlimited washing rounds, but it requires set-up of complicated microfluidics and it is time-consuming to wash previous round of imagers followed by incubation of current round of imagers to ensure signal fidelity. In contrast, the thermal-plex method presented here only requires a simple temperature control unit and designed DNA thermal probes.

The signal channel number of thermal-plex has the potential to be highly expanded. The current channel number is limited by the temperature bandwidth of the DNA thermal probe melting process (8-10 degrees typically) to ensure minimal signal crosstalk between the neighboring channels. By incorporating the cooperativity⁹ into the design of thermal probes, the bandwidth can be narrowed down to 3-4 degrees potentially, which will double or triple the current channel numbers.

Furthermore, even just with the current five thermal channels, combinatorial encoding of both the fluorophore channel and the thermal channel is efficient to achieve hundreds or even thousands of RNA profiling in a single cell ^{2, 3}. The thermal plex is a universal imaging method that is adaptable to current other imaging platform, such as buffer exchanged based multiplexed imaging, expansion microscopy, and super resolution microscopy. The inventors have illustrated the application in RNA profiling in situ. The application of thermal-plex for in situ immunofluorescence imaging^{10, 11} and chromosome imaging^{12, 13} is straightforward to achieve in the next steps.

Given the convenience and robustness of thermal-plex for multiplexed fluorescent imaging, it will enable many direct applications in the spatial biology area.

### REFERENCES

1. Lewis, S. M. et al. Spatial omics and multiplexed imaging to explore cancer biology. Nature methods, 1-16 (2021).
2. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
3. Eng, C. L. et al. Transcriptome-scale super-resolved imaging in tissues by RNA seqFISH. Nature 568, 235-239 (2019).
4. Beliveau, B. J. et al. OligoMiner provides a rapid, flexible environment for the design of genome-scale oligonucleotide in situ hybridization probes. Proc Natl Acad Sci U S A 115, E2183-E2192 (2018).
5. Ye, J., McGinnis, S. & Madden, T.L. BLAST: improvements for better sequence analysis. Nucleic Acids Res 34, W6-9 (2006).
6. Lubeck, E. & Cai, L. Single-cell systems biology by super-resolution imaging and combinatorial labeling. Nat Methods 9, 743-748 (2012).
7. Lin, C. et al. Submicrometre geometrically encoded fluorescent barcodes self-assembled from DNA. Nat Chem 4, 832-839 (2012).
8. Jungmann, R. et al. Multiplexed 3D cellular super-resolution imaging with DNA-PAINT and Exchange-PAINT. Nat Methods 11, 313-318 (2014).
9. Hunter, C. A. & Anderson, H.L. What is cooperativity? Angew Chem Int Ed Engl 48, 7488-7499 (2009).
10. Saka, S.K. et al. Immuno-SABER enables highly multiplexed and amplified protein imaging in tissues. Nat Biotechnol 37, 1080-1090 (2019).
11. Goltsev, Y. et al. Deep Profiling of Mouse Splenic Architecture with CODEX Multiplexed Imaging. Cell 174, 968-981 e915 (2018).
12. Wang, S. et al. Spatial organization of chromatin domains and compartments in single chromosomes. Science 353, 598-602 (2016).
13. Bintu, B. et al. Super-resolution chromatin tracing reveals domains and cooperative interactions in single cells. Science 362 (2018).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for detecting a target molecule in a sample, the method comprising:
a. binding a first nucleic acid strand to a target molecule, wherein the first nucleic acid strand comprises a first hybridization domain and is linked to a target binding agent capable of binding with the target molecule;
b. contacting a probe set with the target molecule from step (a), wherein the probe set comprises:
i. a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and
ii. a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and
wherein a melting temperature of the third strand hybridizing with the second strand is lower than a melting temperature of the second strand hybridizing with the first strand, and
wherein a wherein a melting temperature of the third strand hybridizing with the second strand is lower than a melting temperature of the first strand binding with the target molecule; and
c. analyzing the binding of the probe-set to the target by assessing a detectable signal produced as a function of temperature.

2. A method for multiplex detecting of target molecules in a sample, the method comprising:
a. binding a first nucleic acid strand to a plurality of target molecules, wherein each first nucleic acid strand comprises a first hybridization domain and is linked to a target binding agent capable of binding with the target molecule, and wherein the first hybridization domain of the first nucleic acid strands linked to different target binding agents are different;
b. contacting a plurality of detection probe sets with the target molecules from step (a), wherein each detection probe set comprises:
i. a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and
ii. a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and
wherein in each probe set a melting temperature of the third strand hybridizing with the second strand is lower than a melting temperature of the second strand hybridizing with the first strand, and
wherein in each probe set a melting temperature of the third strand hybridizing with the second strand is lower than a melting temperature of the first strand binding with the target molecule, and
wherein the melting temperature of the third strand hybridizing with the second strand in at least one probe set is different from the melting temperature of the third strand hybridizing with the second strand in at least one other probe set, and a melting temperature of the second strand in the probe set having the third strand with the lower melting temperature is about same or lower than a melting temperature of the second strand in the probe set having the third strand with the higher melting temperature; and
c. analyzing the binding of the probe-sets to the targets by assessing a detectable signal produced as a function of temperature.

3. The method of claim 2, wherein the melting temperature of the third strand hybridizing with the second strand in at least one probe set is at least 5° C. lower than the melting temperature of the third strand hybridizing with the second strand in at least one other probe set.

4. The method of any one of claims 2-3, wherein the reporter molecule in at least one probe set is different from the reporter molecule in at least one other probe set.

5. The method of any one of claims 2-3, wherein reporter molecules in the different probe sets are the same.

6. The method of any one of claims 1-5, wherein the target binding agent is selected from the group consisting of nucleic acids, proteins, peptides, peptidomimetics, amino acids, disaccharides, trisaccharides, oligosaccharides, polysaccharides, lipopolysaccharides, lectins, nucleosides, nucleotides, vitamins, steroids, hormones, cofactors, receptors and receptor ligands.

7. The method of claim 6, wherein the target binding agent is a nucleic acid, antibody, antigen binding fragment of an antibody, antibody mimetic, receptor, or a ligand for a receptor 8 The method of claim 7, wherein the target binding molecule is a nucleic acid.

9. The method of claim 7, wherein the target binding molecule is an antibody or antigen binding fragment of an antibody.

10. The method of any one of claims 1-9, wherein the melting temperature of the third strand hybridizing with the second strand is at least 5° C. lower than the melting temperature of the second strand hybridizing with the first strand.

11. The method of any one of claims 1-10, wherein the melting temperature of the third strand hybridizing with the second strand is at least 5° C. lower than the melting temperature of the first strand binding with the target molecule.

12. The method of any one of claims 1-11, wherein the melting temperature of the second strand hybridizing with the first strand is at least 5° C. lower than the melting temperature of the first strand binding with the target molecule.

13. The method of any one of claims 1-12, wherein at least one of the first, second or third strand comprises a nucleic acid modification.

14. The method of any one of claims 1-13, wherein the first hybridization domain of the first strand and/or the second hybridization domain of the second strand comprises a duplex stabilizing modification.

15. The method of any one of claims 1-14, wherein the third hybridization domain of the second strand and/or the fourth hybridization domain comprises a duplex destabilizing modification.

16. The method of any one of claims 1-15, wherein the reporter molecule and the quencher molecule are a FRET pair.

17. The method of any one of claims 1-16, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

18. The method of any one of claims 1-17, wherein the quencher molecule is a dark quencher.

19. The method of any one of claims 1-18, wherein the first strand further comprises a reporter domain linked to the target binding domain.

20. The method of claim 19, wherein the probe set further comprises a fourth nucleic acid strand, wherein the fourth nucleic acid strand comprises a fifth hybridization domain, wherein the fifth hybridization domain comprises a nucleotide sequence substantially complementary to a nucleotide sequence of the reporter domain of the first strand, and optionally, the fourth strand comprises a reporter molecule.

21. The method of any one of claims 1-20, wherein said analyzing the binding of the probe-set to the target comprises detecting the detectable signal at a first temperature and at a second temperature, wherein one of the first or second temperature is lower than the melting temperature of the third strand hybridizing with the second strand and the other of the first or second temperature is higher than the melting temperature of the third strand hybridizing with the second strand.

22. The method of any one of claims 1-21, wherein the target molecule is selected from the group consisting of nucleic acids, proteins, saccharides, lipids, small molecules, and antigens.

23. The method of claim 22, wherein the target molecule is a nucleic acid.

24. The method of claim 23, wherein the target molecule is RNA.

25. The method of claim 23, wherein the target molecule is DNA.

26. The method of claim 22, wherein the target molecule is a protein.

27. The method of claim 22, wherein the target molecule is a primary or secondary metabolite.

28. The method of any one of claims 1-27, wherein the target molecule is a biomolecule.

29. The method of any one of claims 1-28, wherein the target molecule is in a cell.

30. The method of claim 29, wherein the cell is a fixed cell.

31. A probe set for detecting a target molecule, the probe set comprising i. a first nucleic acid strand comprising a first hybridization domain and linked to a target binding agent capable of binding with the target molecule;
ii. a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and
iii. a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon.

32. A probe set for multiplex detection of target molecule, comprising a plurality of detection probe sets, wherein each detection probe set comprises:
i. a first nucleic acid strand comprising a first hybridization domain and lined to a target binding agent capable of binding with the target molecule, and wherein the first hybridization domain of the first nucleic acid strands linked to different target binding agents are different;
ii. a second nucleic acid strand comprising a second hybridization domain linked to a third hybridization domain, wherein the second hybridization domain comprises a nucleotide sequence substantially complementary to a nucleotide sequence of the first hybridization domain, and wherein the second nucleic acid strand comprises a reporter molecule capable of producing a detectable signal; and
iii. a third nucleic acid strand comprising a fourth hybridization domain, wherein the fourth hybridization domain comprises a nucleotide sequence substantially complementary to a nucleotide sequence of the third hybridization domain, wherein the third nucleic acid strand comprises a quencher molecule, and wherein the quencher molecule quenches the detectable signal from the reporter molecule when the nucleic acid probe is not hybridized to the amplicon, and
wherein a melting temperature of the third strand hybridizing with the second strand is lower than a melting temperature of the second strand hybridizing with the first strand, and
wherein a wherein a melting temperature of the third strand hybridizing with the second strand is lower than a melting temperature of the first strand binding with the target molecule, and
wherein the melting temperature of the third strand hybridizing with the second strand in at least one probe set is different from the melting temperature of the third strand hybridizing with the second strand in at least one other probe set, and
a melting temperature of the second strand in the probe set having the third strand with the lower melting temperature is about same or lower than a melting temperature of the second strand in the probe set having the third strand with the higher melting temperature.

33. The probe set of claim 3, wherein the melting temperature of the third strand hybridizing with the second strand in at least one probe set is at least 5° C. lower than the melting temperature of the third strand hybridizing with the second strand in at least one other probe set.

34. The probe set of any one of claims 32-33, wherein reporter molecules in the different probe sets are the same.

35. The probe set of any one of claims 32-34, wherein the reporter molecule in at least one probe set is different from the reporter molecule in at least one other probe set.

36. The probe set of any one of claims 31-35, wherein the target binding agent is selected from the group consisting of nucleic acids, proteins, peptides, peptidomimetics, amino acids, disaccharides, trisaccharides, oligosaccharides, polysaccharides, lipopolysaccharides, lectins, nucleosides, nucleotides, vitamins, steroids, hormones, cofactors, receptors and receptor ligands.

37. The probe set of claim 36, wherein the target binding agent is a nucleic acid, antibody, antigen binding fragment of an antibody, antibody mimetic, receptor, or a ligand for a receptor

38. The probe set of claim 37, wherein the target binding molecule is a nucleic acid.

39. The probe set of claim 37, wherein the target binding molecule is an antibody or antigen binding fragment of an antibody.

40. The probe set of any one of claims 31-39, wherein the melting temperature of the third strand hybridizing with the second strand is at least 5° C. lower than the melting temperature of the second strand hybridizing with the first strand.

41. The probe set of any one of claims 31-40, wherein the melting temperature of the third strand hybridizing with the second strand is at least 5° C. lower than the melting temperature of the first strand binding with the first strand.

42. The probe set of any one of claims 31-41, wherein at least one of the first, second or third strand comprised a nucleic acid modification.

43. The probe set of any one of claims 31-42, wherein the first hybridization domain of the first strand and/or the second hybridization domain of the second strand comprises a duplex stabilizing modification.

44. The probe set of any one of claims 31-43, wherein the third hybridization domain of the second strand and/or the fourth hybridization domain comprises a duplex destabilizing modification.

45. The probe set of any one of claims 31-44, wherein the reporter molecule and the quencher molecule are a FRET pair.

46. The probe set of any one of claims 31-45, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

47. The probe set of any one of claims 31-46, wherein the quencher molecule is a dark quencher.

48. The probe set of any one of claims 31-47, wherein the first strand further comprises a reporter domain linked to the target binding domain.

49. The probe set of any one of claims 31-48, wherein the probe set further comprises a fourth nucleic acid strand, wherein the fourth nucleic acid strand comprises a fifth hybridization domain, wherein the fifth hybridization domain comprises a nucleotide sequence substantially complementary to a nucleotide sequence of the reporter domain of the first strand, and optionally, the fourth strand comprises a reporter molecule.

50. A kit for detecting a target molecule, the kit comprising a probe-set of any one of claims 31-49.
